(19) United States
(12) Patent Application Publication
(10) Pub. No.: US 2021/0023029 A1
(43) Pub. Date: Jan. 28, 2021

(54) EDASALONEXENT DOSING REGIMEN FOR TREATING MUSCULAR DYSTROPHY

(71) Applicant: Catabasis Pharmaceuticals, Inc., Boston, MA (US)

(72) Inventors: Andrew J. Nichols, Marblehead, MA (US); Michael Perlman, Needham, MA (US)

(21) Appl. No.: 16/761,783

(22) PCT Filed: Nov. 5, 2018
(86) PCT No.: PCT/US2018/059283

§ 371 Date: May 5, 2020

Related U.S. Application Data
(60) Provisional application No. 62/581,981, filed on Nov. 6, 2017.

(51) Int. Cl. A61K 31/166; A61P 21/00; A61K 9/48

(52) U.S. Cl. CPC A61K 31/166; A61P 21/00; A61K 9/0053; A61K 9/4866; A61K 9/4858

(57) ABSTRACT

The invention provides methods and compositions for treating a muscular dystrophy, e.g., Duchenne muscular dystrophy (DMD), in a subject, with a fatty acid acetylated salicylate, e.g., edasalonexent, effective to achieve a threshold plasma concentration of the fatty acid acetylated salicylate in the subject, e.g., a threshold plasma concentration of at least about 20 ng/ml for least 12 hours in a 24 hour period.

### CROSS-REFERENCE TO RELATED APPLICATIONS

This application claims priority to and the benefit of U.S. Provisional Patent Application No. 62/581,981 filed Nov. 6, 2017, the contents of which are incorporated by reference herein in their entirety.

### FIELD OF THE INVENTION

The invention relates to the field of muscular dystrophy, in particular, methods for treating Duchenne muscular dystrophy (DMD), in particular, dosing regimens for treating DMD with edasalonexent, a fatty acid salicylate conjugate.

### BACKGROUND

Duchenne muscular dystrophy (DMD) is a rare, serious, life-threatening, degenerative neuromuscular disease with a recessive X-linked inheritance. Caused by mutations in the dystrophin gene, DMD is characterized by the absence, or near absence, of functional dystrophin protein, leading to the progressive deterioration of skeletal muscle function from early childhood. Despite improvements in the standard of care, such as the use of glucocorticoids, DMD remains an ultimately fatal disease, with patients usually dying of respiratory or cardiac failure by thirty years of age.

The absence of functional dystrophin in DMD results in muscle fibers susceptible to mechanical stress, muscle damage, inflammation of muscle cells, and reduced ability to regenerate muscle tissue. NF-κB is a family of transcriptional factors that is activated in DMD. The NF-κB family of transcriptional factors include p50 (NF-κB1), p52 (NF-κB2), p65 (RelA), c-Rel and RelB. These nuclear factors are maintained in an inactive state in the cytoplasm as a complex by a NF-κB inhibitory factor IκB, such as IκBα, IκBβ, and IκB. The inactive NF-κB complex is released from the cytoplasm by phosphorylation of the IκB protein through kinases such as IKKβ. The kinases regulating NF-κB activity are activated by immune responses or cellular stresses. Thus, in the cytoplasmic NF-κB complex such as IkB/p65/p50, IkB becomes phosphorylated through kinases such as IKKβ and releases dimeric pairs of NF-κB to the nucleus such as p65/p50. In the nucleus, NF-κB regulates genetic expression of proinflammatory factors such as cytokines like TNFα, IL-6, and IL-1β in addition to enzymes such as cyclooxygenase-2 (COX-2), one of the enzymes that converts arachidonic acid to prostaglandin H2 (PGH2). These factors induce inflammation in various tissues. In addition, depending upon the cellular context and the NF-κB nuclear factors released, NF-κB can cause the expression of anti-inflammatory genes. Though these pathways are essential to organism survival and adaptation, chronic activation of the NF-κB system results in uncontrolled inflammatory pathology. Such is the case in dystrophin-deficient muscle, where chronic activation of NF-κB occurs in the muscle of dystrophic mice and DMD patients.

In DMD patients, the activation of NF-κB typically is observed in muscle tissue prior to the onset of other clinical manifestations. In addition, the immune cells and degenerating muscle fibers of DMD patients show elevated levels of activated NF-κB. Evidence also suggests that mechanical stress activates NF-κB in muscle and drives NF-κB mediated inflammation. More rapid deterioration of muscle is observed in muscles with increased mechanical stress and inflammation, for example, quadriceps and hamstrings.

Edasalonexent is an orally bioavailable NF-κB inhibitor that comprises a polyunsaturated fatty acid (PUFA) and salicylic acid, which individually inhibit the activation of NF-κB, conjugated together by a linker that is only susceptible to hydrolysis by intracellular fatty acid hydrolase. These compounds have been shown to inhibit NF-κB activation in vitro, and that long-term treatment improves the phenotype of both the mdx mouse and golden retriever muscular dystrophy (GRMD) dog models of DMD (Hammers et al. (2016) JCI INSIGHT 1(21):e90341).

Despite advances to date, there remains a need for improved methods for treating muscular dystrophy, such as DMD, in patients, including methods for treating DMD with NF-kB inhibitors.

### SUMMARY

The invention provides methods and compositions for treating a muscular dystrophy, e.g., Duchenne muscular dystrophy (DMD). The invention is based, in part, upon the discovery that when treating DMD in a subject with a fatty acid acetylated salicylate, e.g., edasalonexent, efficacy is driven by the amount of time that the fatty acid acetylated salicylate is at or above a threshold plasma concentration in the subject, rather than the maximum concentration of the fatty acid acetylated salicylate in the plasma or total exposure to the fatty acid acetylated salicylate.

Accordingly, in one aspect, the invention provides a method of treating muscular dystrophy, e.g., Duchenne muscular dystrophy (DMD), in a subject in need thereof. The method comprises administering to the subject a dosing regimen of a compound having the structure of Formula I,

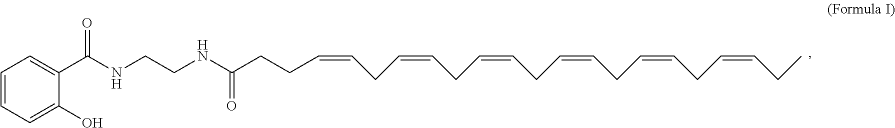 [SMILES: CC/C=C\C/C=C\C/C=C\C/C=C\C/C=C\C/C=C\CCC(=O)NCCNC(=O)c1ccccc1O]

or a pharmaceutically acceptable salt thereof, effective to achieve a threshold plasma concentration of the compound in the subject of at least about 20 ng/ml for least 12 hours in a 24 hour period. In certain embodiments, the threshold plasma concentration is from about 20 ng/ml to about 200 ng/ml. In certain embodiments, the compound is at or above the threshold concentration for at least about 13 hours, about 14 hours, about 15 hours, about 16 hours, about 17 hours, about 18 hours, about 19 hours, about 20 hours, about 21 hours, about 22 hours, about 23 hours, or about 24 hours in a 24 hour period.

In certain embodiments, the dosing regimen comprises one, two or three doses of the compound per day. In certain embodiments, each dose comprises from about 25 mg/kg to about 100 mg/kg of the compound. In certain embodiments, each dose comprises from about 25 mg/kg to about 50 mg/kg of the compound. In certain embodiments, each dose comprises from about 20 mg/kg to about 40 mg/kg of the compound. In certain embodiments, the total daily dosage comprises from about 100 mg/kg to about 200 mg/kg, or from about 100 mg/kg to about 150 mg/kg, e.g., 100 mg/kg or 133 mg/kg. In certain embodiment, the total daily dosage comprises about 100 mg/kg. In certain embodiments, the total daily dosage comprises from about 90 mg/kg to about 110 mg/kg. In certain embodiments, the total daily dosage comprises 100 mg/kg±5%, 100 mg/kg±10%, 100 mg/kg±15%, or 100 mg/kg±20% of the compound.

In certain embodiments, the dosing regimen comprises three doses per day. In certain embodiments, the three doses comprise equal amounts of the compound, e.g., each dose comprises from about 25 mg/kg to about 50 mg/kg of the compound, or e.g., each dose comprises about 33 mg/kg of the compound.

In certain embodiments, the first dose and the second dose comprise a smaller amount of the compound than the third dose, e.g., the first dose and the second dose comprise about half the amount of the compound as the third dose. For example, in certain embodiments, the first dose and the second dose comprise from about 25 mg/kg to about 50 mg/kg of the compound, and the third dose comprises from about 50 mg/kg to about 100 mg/kg of the compound, e.g., the first dose and the second dose comprise about 33 mg/kg of the compound and the third dose comprises about 67 mg/kg of the compound.

In certain embodiments, at least one of the three doses is a different amount from the other two doses, e.g., each dose comprises from about 25 mg/kg to about 50 mg/kg of the compound, or e.g., each dose comprises from about 20 mg/kg to about 40 mg/kg of the compound. In one embodiment, two doses are the same and one is different. In one embodiment, all three doses are different.

In certain embodiments, the three doses are equal and are administered in dosage forms that contain 250 mg or 100 mg of the compound of Formula I. In certain other embodiments, the three dosages are not equal and are administered in dosage forms that contain 250 mg or 100 mg of the compound of Formula I, e.g., two are equal and one is different or e.g., each dose is different. In certain embodiments, the three doses equal a total daily dose of 100 mg/kg±5%, 100 mg/kg±10%, 100 mg/kg±15%, or 100 mg/kg±20% of the compound. In certain embodiments, two doses are 750 mg and one dose is 500 mg. In certain embodiments, the total daily dose does not exceed 6,000 mg.

In certain embodiments, the first dose is administered in the morning, the second dose is administered at mid-day, and the third dose is administered in the evening. In certain embodiments, each dose is administered with food, e.g., at the time of a meal. For example, in certain embodiments, the first dose is administered at the time of breakfast, the second dose is administered at the time of lunch, and the third dose is administered at the time of dinner. In certain embodiments, two doses are administered with breakfast and dinner that are larger than the dose administered with lunch. In certain embodiments, the dose is administered with food or a meal containing at least 8 g of fat.

In certain embodiments, the compound is administered in a pharmaceutical composition, e.g., a composition comprising 50-70% by weight of the compound. The composition may, e.g., further comprise one or more of glyceryl monooleate (type 40), polysorbate 80, polyethylene glycol 400, or DL-α-tocopherol. In certain embodiments, the composition is formulated as a capsule. In certain embodiments, the compound is administered orally.

In certain embodiments, the method reduces inflammation in quadriceps muscle by at least 20%, and/or reduces fibrosis in quadriceps muscle by at least 20%.

In another aspect, the invention provides a pharmaceutical composition comprising 50-70% by weight of a compound having the structure of Formula I,

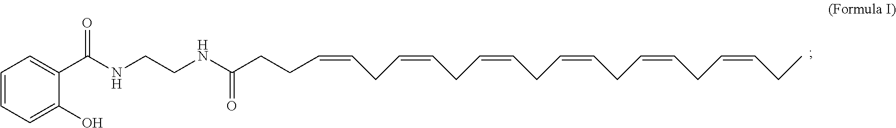 [SMILES: CC/C=C\C/C=C\C/C=C\C/C=C\C/C=C\C/C=C\CCC(=O)NCCNC(=O)c1ccccc1O]

or a pharmaceutically acceptable salt thereof, and optionally one, two, three, or four of: a solvent or diluent (e.g., glyceryl monooleate (type 40)); a surfactant (e.g., a nonionic surfactant, e.g., polysorbate 80); a co-solvent (e.g., polyethylene glycol 400); and an anti-oxidant (e.g., DL-α-tocopherol). In certain embodiments, the solvent or diluent is glyceryl monooleate (type 40). In certain embodiments, the surfactant is a non-ionic surfactant, e.g., polysorbate 80. In certain embodiments, the so-solvent is polyethylene glycol. In certain embodiments, the anti-oxidant is DL-α-tocopherol.

Various aspects and embodiments of the invention are described in more detail below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise.

### BRIEF DESCRIPTION OF THE FIGURES

FIG. 1 shows line graphs of the concentration level of edasalonexent in the plasma and skeletal muscle of C57BL/6 mice that received a dose of 1.5% edasalonexent in their diet.

FIG. 2 is a line graph showing the plasma concentration level of edasalonexent in C57BL/6 mice that received a single oral daily dose of 450 mg/kg edasalonexent, three oral daily doses of 150 mg/kg edasalonexent (for a total daily dose of 450 mg/kg), or two oral daily doses of 150 mg/kg edasalonexent and one oral daily dose of 300 mg/kg edasalonexent (for a total daily dose of 600 mg/kg).

FIG. 3 shows bar graphs depicting quadriceps muscle inflammation (left) and fibrosis (right) in mdx mice that received a dose of 1% edasalonexent in their diet.

FIG. 4 shows bar graphs depicting quadriceps muscle inflammation (left) and fibrosis (right) in mdx mice that received a single oral daily dose of 450 mg/kg edasalonexent and/or a dose of 1% edasalonexent in their diet.

FIG. 5 shows bar graphs depicting quadriceps muscle inflammation (left) and fibrosis (right) in mdx mice that received a single oral daily dose of 450 mg/kg edasalonexent, three oral daily doses of 150 mg/kg edasalonexent (for a total daily dose of 450 mg/kg), or two oral daily doses of 150 mg/kg edasalonexent and one oral daily dose of 300 mg/kg edasalonexent (for a total daily dose of 600 mg/kg).

FIG. 6 shows edasalonexent plasma concentration for subjects from the MoveDMD® phase 2 trial receiving two 33 mg/kg doses per day (for a total daily dose of 67 mg/kg/day).

FIG. 7 shows edasalonexent plasma concentration for subjects from the MoveDMD® phase 2 trial receiving three 33 mg/kg doses per day (for a total daily dose of 100 mg/kg/day).

FIG. 8 shows modeled edasalonexent plasma concentration levels for dosing regimens including three 33 mg/kg doses per day (for a total daily dose of 100 mg/kg/day), or two 33 mg/kg doses and one 67 mg/kg dose per day (for a total daily dose of 133 mg/kg/day) based on population PK model developed using data from the edasalonexent phase 1 and phase 2 clinical trials.

FIG. 9 shows modeled edasalonexent plasma concentration levels for dosing regimens including three 33 mg/kg doses per day (for a total daily dose of 100 mg/kg/day), or two 33 mg/kg doses (for a total daily dose of 67 mg/kg/day) based on population PK model developed using data from the edasalonexent phase 1 and phase 2 clinical trials.

FIG. 10 is a schematic depiction of the MoveDMD Phase I and Phase II clinical trial design.

FIG. 11 is a bar chart showing the daily exposure of subjects to edasalonexent in ng/mL*hr when exposed to dosages of 33 mg/kg, 67 mg/kg, or 100 mg/kg.

FIG. 12 is a bar chart showing the change in expression levels between Day 1 (prior to edasalonexent treatment) and Day 7 (of edasalonexent treatment) for 24 different NF-κB regulated and inflammation regulated gene transcripts when subjects were exposed to dosages of 33 mg/kg, 67 mg/kg, or 100 mg/kg. Each column represents data for an individual gene transcript.

FIG. 13 is a graph showing the average change in expression levels between Day 1 (prior to edasalonexent treatment) and Day 7 (of edasalonexent treatment) for 24 different NF-κB regulated and inflammation regulated gene transcripts versus the mean C_{trough} (ng/mL) for three dose groups (33 mg/kg/day, 67 mg/kg/day, and 100 mg/kg/day).

FIG. 14 is a line graph showing the average rate of change of North Star Ambulatory Assessment scores for study subjects in the 100 mg/kg/day edasalonexent dosing group over a 36 week control period followed by the 60 week treatment period.

FIG. 15 is a line graph showing the average rate of change in 10-Meter Walk/Run times for study subjects in the 100 mg/kg/day edasalonexent dosing group over a 36 week control period followed by the 60 week treatment period.

FIG. 16 is a line graph showing the average rate of change in 4-stair climb times for study subjects in the 100 mg/kg/day edasalonexent dosing group over a 36 week control period followed by the 60 week treatment period.

FIG. 17 is a line graph showing the average rate of change in Time to Stand times for study subjects in the 100 mg/kg/day edasalonexent dosing group over a 36 week control period followed by the 60 week treatment period.

FIG. 18 is a bar graph showing the annualized rate of change in MRI-T2 values for study subjects in the 100 mg/kg/day edasalonexent dosing group over a 36 week control period followed by a 48 week treatment period.

FIG. 19 is a table showing the change in fat fraction values for study subjects in the 100 mg/kg/day edasalonexent dosing group over a 36 week control period followed by a 48 week treatment period.

FIGS. 20A-D are line graphs showing heart rate change from baseline in beats per minute (FIG. 20A), change in height percentile (FIG. 20B), change in body mass index (BMI) percentile (FIG. 20C), and change in weight percentile (FIG. 20D) for study subjects over the 60 week study period.

### DETAILED DESCRIPTION

The invention provides methods and compositions for treating a muscular dystrophy, e.g., Duchenne muscular dystrophy (DMD). The invention is based, in part, upon the discovery that when treating DMD in a subject with a fatty acid acetylated salicylate, e.g., edasalonexent, efficacy is driven by the amount of time that the fatty acid acetylated salicylate is at or above a threshold plasma concentration in the subject, rather than the maximum concentration of the fatty acid acetylated salicylate in the plasma or total exposure to the fatty acid acetylated salicylate. Accordingly, in one aspect, the invention provides a method of treating muscular dystrophy, e.g., Duchenne muscular dystrophy (DMD), in a subject in need thereof. The method comprises administering to the subject a dosing regimen of a compound having the structure of Formula I,

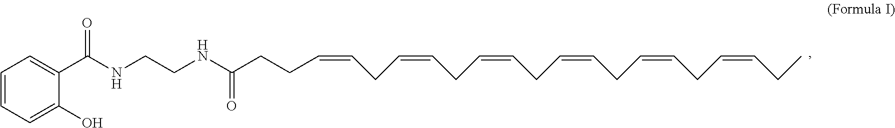 [SMILES: CC/C=C\C/C=C\C/C=C\C/C=C\C/C=C\C/C=C\CCC(=O)NCCNC(=O)c1ccccc1O]

or a pharmaceutically acceptable salt thereof, effective to achieve a threshold plasma concentration of the compound in the subject of at least about 20 ng/ml for least 12 hours in a 24 hour period.

In certain embodiments, the threshold plasma concentration is from about 20 ng/ml to about 200 ng/ml. For example, in certain embodiments, the threshold plasma concentration is from about 20 ng/ml to about 200 ng/ml, from about 20 ng/ml to about 175 ng/ml, from about 20 ng/ml to about 150 ng/ml, from about 20 ng/ml to about 125 ng/ml, from about 20 ng/ml to about 100 ng/ml, from about 20 ng/ml to about 75 ng/ml, from about 20 ng/ml to about 50 ng/ml, from about 20 ng/ml to about 25 ng/ml, from about 25 ng/ml to about 200 ng/ml, from about 25 ng/ml to about 175 ng/ml, from about 25 ng/ml to about 150 ng/ml, from about 25 ng/ml to about 125 ng/ml, from about 25 ng/ml to about 100 ng/ml, from about 25 ng/ml to about 75 ng/ml, from about 25 ng/ml to about 50 ng/ml, from about 50 ng/ml to about 200 ng/ml, from about 50 ng/ml to about 175 ng/ml, from about 50 ng/ml to about 150 ng/ml, from about 50 ng/ml to about 125 ng/ml, from about 50 ng/ml to about 100 ng/ml, from about 50 ng/ml to about 75 ng/ml, from about 75 ng/ml to about 200 ng/ml, from about 75 ng/ml to about 175 ng/ml, from about 75 ng/ml to about 150 ng/ml, from about 75 ng/ml to about 125 ng/ml, from about 75 ng/ml to about 100 ng/ml. from about 100 ng/ml to about 200 ng/ml, from about 100 ng/ml to about 175 ng/ml, from about 100 ng/ml to about 150 ng/ml, from about 100 ng/ml to about 125 ng/ml, from about 125 ng/ml to about 200 ng/ml, from about 125 ng/ml to about 175 ng/ml, from about 125 ng/ml to about 150 ng/ml, from about 150 ng/ml to about 200 ng/ml, from about 150 ng/ml to about 175 ng/ml, or from about 175 ng/ml to about 200 ng/ml. In certain embodiments, the threshold plasma concentration is about 200 ng/ml, about 175 ng/ml, about 150 ng/ml, about 125 ng/ml, about 100 ng/ml, about 75 ng/ml, about 50 ng/ml, about 25 ng/ml, or about 20 ng/ml. The plasma concentration of an active agent described herein, e.g., edasalonexent, may be measured by methods known in the art, including by LC/MS/MS (liquid chromatography/mass spectrometry/mass spectrometry).

In certain embodiments, the compound is at or above the threshold concentration for at least from about 12 hours to about 24 hours, from about 14 hours to about 24 hours, from about 16 hours to about 24 hours, from about 18 hours to about 24 hours, from about 20 hours to about 24 hours, from about 22 hours to about 24 hours, from about 12 hours to about 22 hours, from about 14 hours to about 22 hours, from about 16 hours to about 22 hours, from about 18 hours to about 22 hours, from about 20 hours to about 22 hours, from about 12 hours to about 20 hours, from about 14 hours to about 20 hours, from about 16 hours to about 20 hours, from about 18 hours to about 20 hours, from about 12 hours to about 18 hours, from about 14 hours to about 18 hours, from about 16 hours to about 18 hours, from about 12 hours to about 16 hours, from about 14 hours to about 16 hours, or from about 12 hours to about 14 hours in a 24 hour period.

In certain embodiments, the compound is at or above the threshold concentration for at least about 13 hours, about 14 hours, about 15 hours, about 16 hours, about 17 hours, about 18 hours, about 19 hours, about 20 hours, about 21 hours, about 22 hours, about 23 hours, or about 24 hours in a 24 hour period.

Various features and aspects of the invention are discussed in more detail below.

### I. Definitions

To facilitate an understanding of the present invention, a number of terms and phrases are defined below.

As used herein, the terms “subject” and “patient” are used interchangeably and refer to an organism to be treated by the methods and compositions of the present invention. Such organisms are preferably a mammal (e.g., human, mouse, rat, guinea pig, dog, cat, horse, cow, pig, or non-human primate, such as a monkey, chimpanzee, baboon, and rhesus), and more preferably, a human.

As used herein, the phrases “effective amount” and “therapeutically effective amount” refer to the amount of a compound (e.g., a compound described herein) sufficient to effect beneficial or desired results. An effective amount can be administered in one or more administrations, applications or dosages and is not intended to be limited to a particular formulation or administration route.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see Martin, Remington's Pharmaceutical Sciences, 15th Ed., Mack Publ. Co., Easton, Pa. (1975).

As used herein, the term “pharmaceutically acceptable salt” refers to any salt of an acidic or a basic group that may be present in a disclosed compound, which salt is compatible with pharmaceutical administration. As is known to those of skill in the art, “salts” of the disclosed compounds may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic and benzenesulfonic acid. Other acids, such as oxalic, while not in themselves pharmaceutically acceptable, may be employed in the preparation of salts useful as intermediates in obtaining the compounds described herein and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺ and NW₄⁺ (where W can be a C₁₋₄ alkyl group), and the like. For therapeutic use, salts of the compounds disclosed herein are contemplated as being pharmaceutically acceptable.

The term “carrier” refers to excipients and diluents, and means a material, composition or vehicle, such as a liquid or solid filler, diluent, excipient, solvent or encapsulating material, involved in carrying or transporting a pharmaceutical agent from one organ, or portion of the body, to another organ, or portion of the body.

As used herein, the term “treating” includes any effect, for example, lessening, reducing, modulating, ameliorating or eliminating, that results in the improvement of the condition, disease, disorder, and the like, or ameliorating a symptom thereof. Treating can be curing, improving, or at least partially ameliorating the disorder. In certain embodiments, treating is curing the disease.

The term “disorder” refers to and is used interchangeably with, the terms “disease,” “condition,” or “illness,” unless otherwise indicated.

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

“Chronic administration,” as used herein, refers to continuous, regular, long-term administration, i.e., periodic administration without substantial interruption. For example, daily, for a period of time of at least several weeks or months or years, for the purpose of treating muscular dystrophy in a patient. For example, weekly, for a period of time of at least several months or years, for the purpose of treating muscular dystrophy in a patient (e.g., weekly for at least six weeks, weekly for at least 12 weeks, weekly for at least 24 weeks, weekly for at least 48 weeks, weekly for at least 72 weeks, weekly for at least 96 weeks, weekly for at least 120 weeks, weekly for at least 144 weeks, weekly for at least 168 weeks, weekly for at least 180 weeks, weekly for at least 192 weeks, weekly for at least 216 weeks, or weekly for at least 240 weeks).

“Periodic administration,” as used herein, refers to administration with an interval between doses. For example, periodic administration includes administration at fixed intervals (e.g., weekly, monthly) that may be recurring.

Unless defined otherwise, all technical and scientific terms used herein have the same meaning as commonly understood by one of ordinary skill in the art to which this invention belongs.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also include the specific quantitative value itself, unless specifically stated otherwise. As used herein, the term “about” refers to a ±10% variation from the nominal value unless otherwise indicated or inferred.

Where a molecular weight is provided and not an absolute value, for example, of a polymer, then the molecular weight should be understood to be an average molecule weight, unless otherwise stated or understood from the context.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

As a general matter, compositions specifying a percentage are by weight unless otherwise specified. Further, if a variable is not accompanied by a definition, then the previous definition of the variable controls.

### II. Edasalonexent and Other NF-κB Inhibitors

An example of a fatty acid acetylated salicylate that can inhibit NF-κB activity and reduce inflammation is edasalonexent, also referred to as CAT-1004 (Milne et al. (2014) NEUROMUSCULAR DISORDERS 24(9):825). Edasalonexent, [N-(2-[(4Z,7Z,10Z,13Z,16Z,19Z)-docosa-4,7,10,13,16,19-hexaenamido]ethyl)-2-hydroxybenzamide], is a small molecule in which salicylic acid and docosahexaenoic acid (DHA) are covalently conjugated through an ethylenediamine linker and that is designed to synergistically leverage the ability of both of these compounds to inhibit NF-κB. Edasalonexent is assigned CAS Registry No. 1204317-86-1 and has the structure of Formula I:

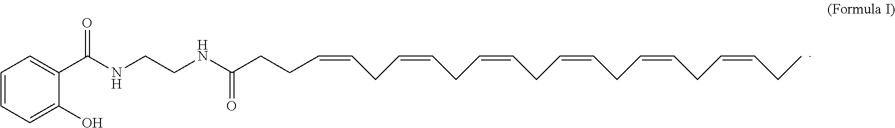 [SMILES: CC/C=C\C/C=C\C/C=C\C/C=C\C/C=C\C/C=C\CCC(=O)NCCNC(=O)c1ccccc1O]

Edasalonexent has been shown to enhance muscle regeneration, reduce muscle degeneration and inflammation, and preserve muscle function in mdx mice (Milne, J. et al., (2014) supra). In long-term studies on mdx mice, edasalonexent treatment results in improved diaphragm function and increased cumulative run distance (Milne, J. et al., (2014) supra). In a dog model of DMD, edasalonexent decreases NF-κB activity as evidenced by reduced binding of the p65 subunit to DNA and reduced secretion of the inflammatory mediator TNF-α. In humans, administration of edasalonexent results in a decrease of biomarkers of inflammation in whole blood. In healthy adult humans, edasalonexent treatment also lowers levels of the p65 subunit of NF-κB compared to treatment with a placebo or with salicylate and omega-3 DHA as separate molecules

It is contemplated that edasalonexent may be replaced by a structural homolog known as CAT-1041, which is structurally similar to edasalonexent but DHA is replaced with eicosapentaenoic acid (EPA). In long-term studies on mdx mice, CAT-1041 treatment preserves muscle function, increases skeletal muscle weight, and reduces muscle fibrosis. CAT-1041 may also reduce cardiomyopathy in mdx mice.

An exemplary synthesis of edasalonexent is described in International Patent Publication No. WO2010/006085A1, and is depicted as follows:

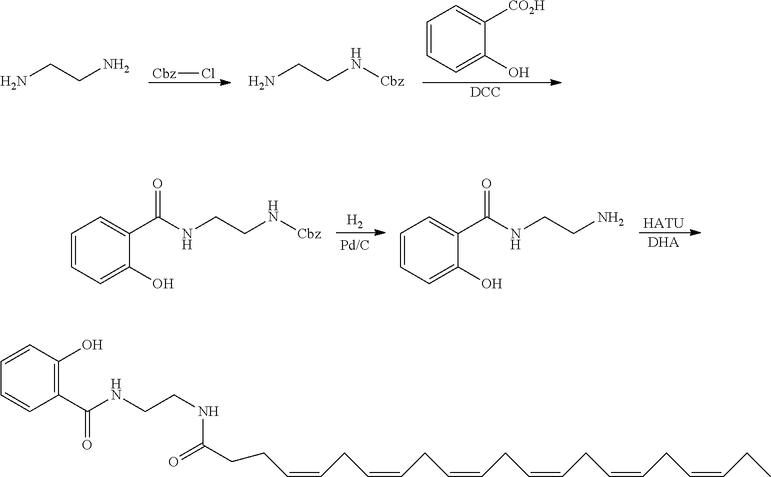

Briefly, ethylenediamine is dissolved in water containing bromoaresal green as an indicator. Methane sulfonic acid in water is added until a blue to pale yellow color transition is just achieved. The solution is diluted with ethanol and vigorously stirred. To the mixture is added the solution of Cbz-CI in dimethoxy ethane and 50% w/v aqueous AcOK at 20° C. simultaneously to maintain the pale yellow-green color of the indicator. After the additions are complete the mixture is stirred and concentrated at low temperature under vacuum to remove the volatiles. The residue is shaken with water and filtered. The filtrate is then washed with toluene, basified with excess 40% aqueous NaOH and extracted with toluene. The organic layer is washed with brine, dried over Na₂SO₄ and evaporated to give benzyl 2-aminoethylcarbamate as an oil.

To a mixture of benzyl 2-aminoethylcarbamate, imidazole, salicylic acid in ethyl acetate is added a solution of DCC in ethyl acetate. The mixture is stirred and filtered. The solution is concentrated under reduced pressure and the crude product is purified by silica chromatography to afford benzyl 2-(2-hydroxybenzamido)ethylcarbamate as a white solid.

A mixture of benzyl 2-(2-hydroxybenzamido)ethylcarbamate and Pd/C in MeOH is stirred under a H₂ atmosphere. The mixture is filtered and concentrated under reduced pressure. The crude product is purified by silica chromatography to afford N-2-(aminoethyl)2-hydroxybenzamide as a white powder.

To a mixture of N-2-(aminoethyl)2-hydroxybenzamide, DHA and Et₃N in CH₃CN is added HATU. The mixture is stirred and concentrated under reduced pressure. The residue is treated with brine and extracted with EtOAc. The combined organic layers are washed with 1M HCl, brine, 5% NaHCO₃ and brine. The organic solution is dried over MgSO₄ and concentrated under reduced pressure. The crude product is purified by silica chromatography to afford N-(2-docosa-4, 7, 10, 13, 16, 19-hexaenamidoethyl)-2-hydroxybenzamide as light yellow oil. CAT-1041 may be produced by a similar approach except the DHA is replaced with EPA.

### III. Pharmaceutical Compositions

Edasalonexent, and/or CAT-1041, may be formulated with one or more pharmaceutically acceptable carriers to facilitate delivery, for example, oral delivery or subcutaneous delivery.

The pharmaceutical compositions of edasalonexent and/or CAT-1041 can be formulated for administration in solid or liquid form, including those adapted for the following: (1) oral administration, for example, capsules (aqueous or non-aqueous solutions or suspensions), tablets, boluses, powders, granules, and pastes; (2) parenteral administration by, for example, subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) sublingually; (5) transdermally; or (6) nasally.

The compositions can conveniently be presented in unit dosage form and can be prepared by any methods well known in the art of pharmacy. The amount of active ingredient which can be combined with a carrier material to produce a single dosage form will vary depending upon the host being treated, and the particular mode of administration. Compositions can be prepared according to conventional mixing, granulating or coating methods.

The capsules, tablets, or other solid dosage forms of the active ingredient, e.g., edasalonexent, can be prepared with coatings and/or shells, such as enteric coatings and other coatings well known in the pharmaceutical-formulating art. They also can be formulated so as to provide slow or controlled release of the active ingredient therein using, for example, hydroxypropylmethyl cellulose in varying proportions to provide the desired release profile, other polymer matrices, liposomes and/or microspheres. They can be sterilized by, for example, filtration through a bacteria-retaining filter, or by incorporating sterilizing agents in the form of sterile solid compositions which can be dissolved in sterile water, or some other sterile injectable medium immediately before use. These compositions also can optionally contain opacifying agents and can be of a composition that they release the active ingredient(s) only, or preferentially, in a certain portion of the gastrointestinal tract, optionally, in a delayed manner Liquid dosage forms for oral administration of the active ingredient, e.g., edasalonexent, can include pharmaceutically acceptable emulsions, microemulsions, solutions, suspensions, syrups and elixirs.

Illustrative pharmaceutical compositions are capsules, for example, gelatin capsules, or tablets including edasalonexent, described herein, and a pharmaceutically acceptable carrier, such as: a) a solvent or diluent; b) a surfactant; c) a co-solvent; or d) an anti-oxidant. Exemplary solvents or diluents include purified water, triglyceride oils, such as hydrogenated or partially hydrogenated vegetable oil, or mixtures thereof, corn oil, olive oil, sunflower oil, safflower oil, fish oils such as EPA or DHA, their esters or triglycerides or mixtures thereof, or glyceryl monooleate (type 40). Exemplary surfactants include sodium lauryl sulfate, sodium dioctyl sulfosuccinate, polysorbate 80, polysorbate 20, cetyl triethyl ammonium bromide, polyethyelene oxide-polypropylene oxide copolymers, Cremophor EL, Span 80, Span 20, Tween 20, Tween 40, Tween 60, Tween 80, Brij L23, Brij 35, Labrasol, Plurol isostearique, dioctyl sodium sulfosuccinate, PEG-35 castor oil (Macrogolglycerol ricinoleate), PEG-40 hydrogenated castor oil (Macrogolglycerol hydroxystearate). diethylene glycol, monoethyl ether, 1,2-octanediol, Epikuron, 1,2-propanediol, benzyl alcohol, Aerosol OT, dodecylglucoside, cocoamide propylbetaine, phosphatidylcholine, 2-ethyl-1,3-hexanediol, caprylic/capric mono-/di-glycerides, polysorbate, Brij, Tagat, isopropyl alcohol, propanol, glycolipid, Lipoid, sodium monohexylphosphate, propylene glycol, n-butanol, glyceryloleate, polyoxyl 40 fatty acid derivatives, tetraglycol, O-alkylglycerol, dodecylglycerol, tetradecylglycerol, taurodeoxycholate, sucrose monolaurate, sucrose dilaurate, isooctanol, Epikuron, Oramix, 1,2-hexanediol, bis-2-(ethylhexyl)sulfosuccinate, n-propanol, 1,2-propylene glycol, glycerol monooleate, hexanol, sucrose laurate, Plurololeat, hexadecyltrimethylammonium bromide, or decanol. Exemplary co-solvents include polyethylene glycol 400, polyethylene glycol 3350, polyethylene glycol 300, ethyl alcohol, isopropyl alcohol, propylene glycol, butanediol, pentanediol, hexanediol, triethylene glycol, tetraethylene glycol, dipropylene glycol, dibutylene glycol, glycerin, dimethyl isosorbide, tetrahydrofurfuryl alcohol polyethylene glycol ether, N-methyl-2-pyrrolidone, 1-methyl-2-pyrrolidinone, dimethyl sulfoxide, dimethyl acetamide, lactic acid, glycolic acid, methylene chloride, methyl-ethyl-ketone, ethyl acetate, or methylene dimethyl ether. Exemplary anti-oxidants include DL-α-tocopherol, propyl gallate, tertiary butylhydroquinone (tBHQ), butylated hydroxyanisole (BHA) or butylated hydroxytoluene (BHT), sodium sulphite, N-acetylcysteine, ascorbic acid, edetic acid, sodium edetate, L-cysteine, sodium metabisulfite, glutathione, cysteine, captopril, N-acetyl cysteine, glutathione, Na-ascorbate, L-cysteine, Na₂-EDTA, Na₂-EDTA-Ca, methimazole, quercetin, arbutin, aloesin, N-acetylglucoseamine, α-tocopheryl ferulate, MAP (Mg ascorbyl phosphate), sodium benzoate, L-phenylalanine, DMSA (succimer), DPA (D-penicillamine), trientine-HCl, dimercaprol, clioquinol, sodium thiosulfate, TETA, TEPA, curcumin, neocuproine, tannin, cuprizone, sodium hydrogen sulfite, lipoic acid, CB4, CB3, AD4, AD6, AD7, Vitamin E, di-tert-butyl methyl phenols, tert-butyl-methoxyphenols, polyphenols, tocopherols, ubiquinones, or caffeic acid.

Edasalonexent, also can be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of an analog described herein to a form lipid layer encapsulating the analog, as described in U.S. Pat. No. 5,262,564.

Parenteral injectable administration is generally used for subcutaneous, intramuscular or intravenous injections and infusions. Injectables can be prepared in conventional forms, either as liquid solutions or suspensions or solid forms suitable for dissolving in liquid prior to injection.

Present pharmaceutical compositions can contain from about 0.1% to about 80%, from about 5% to about 60%, or from about 1% to about 20% of the active ingredient, e.g., edasalonexent, by weight or volume. In certain embodiments, a pharmaceutical composition of the invention contains from about 50% to about 70% by weight of the active ingredient. For example, a pharmaceutical composition may contain from about 50% to about 65%, about 50% to about 60%, about 50% to about 55%, about 55% to about 70%, about 55% to about 65%, about 55% to about 60%, about 60% to about 70%, about 60% to about 65%, or about 65% to about 70% by weight of the active ingredient, e.g., edasalonexent.

In certain embodiments, a pharmaceutical composition of the invention may comprise, one, two, three or more of: a solvent or diluent (e.g., glyceryl monooleate (type 40)); a surfactant (e.g., a nonionic surfactant, e.g., polysorbate 80); a co-solvent (e.g., polyethylene glycol 400); and an anti-oxidant (e.g., DL-α-tocopherol).

In certain exemplary dosage forms, the composition comprises, per kg, 500-700 g of the active ingredient, 150-250 g of glyceryl monooleate (type 40), 100-200 g of polysorbate 80, 10-70 g polyethylene glycol 400, and 0.5-5 g of DL-α-tocopherol.

In certain exemplary dosage forms, the composition comprises, per kg, 550-650 g of the active ingredient, 175-230 g of glyceryl monooleate (type 40), 130-180 g of polysorbate 80, 20-60 g polyethylene glycol 400, and 1-4 g of DL-α-tocopherol.

In certain exemplary dosage forms, the composition comprises, per kg, 550-625 g of the active ingredient, 170-220 g of glyceryl monooleate (type 40), 130-170 g of polysorbate 80, 20-60 g polyethylene glycol 400, and 1-4 g of DL-α-tocopherol.

In certain exemplary dosage forms, the composition comprises, per kg, 585-615 g of the active ingredient, 180-220 g of glyceryl monooleate (type 40), 140-180 g of polysorbate 80, 20-60 g polyethylene glycol 400, and 1-4 g of DL-α-tocopherol.

In certain exemplary dosage forms, the composition comprises, per kg, 590-610 g of the active ingredient, 180-220 g of glyceryl monooleate (type 40), 140-180 g of polysorbate 80, 20-60 g polyethylene glycol 400, and 1-4 g of DL-α-tocopherol.

In certain exemplary dosage forms, the composition comprises, per kg, 585-650 g of the active ingredient, 180-250 g of glyceryl monooleate (type 40), 140-200 g of polysorbate 80, 20-60 g polyethylene glycol 400, and 1-4 g of DL-α-tocopherol.

### III. Therapeutic Applications

The invention provides methods and compositions for treating muscular dystrophy, e.g., Duchenne muscular dystrophy (DMD), in a subject in need thereof.

In certain embodiments, treatment delays disease progression, slows or reduces the loss of ambulation, reduces muscle inflammation, reduces muscle damage, improves muscle function, reduces loss of pulmonary function, and/or enhances muscle regeneration, or any combination thereof. In certain embodiments, treatment maintains, delays, or slows disease progression. In certain embodiments, treatment maintains ambulation or reduces the loss of ambulation. In certain embodiments, treatment maintains pulmonary function or reduces loss of pulmonary function. In certain embodiments, treatment maintains or increases a stable walking distance in a patient, as measured by, for example, the 6 minute walk test (6MWT). In certain embodiments, treatment maintains or reduces the time to walk/run 10 meters (i.e., the 10 meter walk/run test). In certain embodiments, treatment maintains or reduces the time to stand from supine (i.e., time to stand test). In certain embodiments, treatment maintains or reduces the time to climb four standard stairs (i.e., the four-stair climb test). In certain embodiments, treatment maintains or reduces muscle inflammation in the patient, as measured by, for example, MRI (e.g., MRI of the leg muscles). In certain embodiments, MRI measures T2 and/or fat fraction to identify muscle degeneration. MRI can identify changes in muscle structure and composition caused by inflammation, edema, muscle damage and fat infiltration. In certain embodiments, muscle strength is measured by the North Star Ambulatory Assessment.

In certain embodiments, treatment reduces muscle inflammation, reduces muscle damage, improves muscle function, and/or enhances muscle regeneration. For example, treatment may stabilize, maintain, improve, or reduce inflammation in the subject. Treatment may also, for example, stabilize, maintain, improve, or reduce muscle damage in the subject. Treatment may, for example, stabilize, maintain, or improve muscle function in the subject. In addition, for example, treatment may stabilize, maintain, improve, or enhance muscle regeneration in the subject. In certain embodiments, treatment maintains or reduces muscle inflammation in the patient, as measured by, for example, magnetic resonance imaging (MRI) (e.g., MRI of the leg muscles).

In certain embodiments, treatment is measured by the 6 minute walk test (6MWT). In certain embodiments, treatment is measured by the 10 meter walk/run test. In certain embodiments, treatment results in a reduction or decrease in muscle inflammation in the patient. In certain embodiments, muscle inflammation in the patient is measured by MRI imaging. In certain embodiments, the treatment is measured by the 4-stair climb test. In certain embodiments, treatment is measured by the time to stand test. In certain embodiments, treatment is measured by the North Star Ambulatory Assessment.

In certain embodiments, the patient has lost the ability to rise independently from supine. In certain embodiments, the patient has lost the ability to rise independently from supine at least one year prior to treatment with a method or composition of the invention. In certain embodiments, the patient has lost the ability to rise independently from supine within one year of commencing with a method or composition of the invention. In certain embodiments, the patient has lost the ability to rise independently from supine within two years of commencing treatment with a method or composition of the invention.

In certain embodiments, the patient maintains ambulation for at least 24 weeks after commencing treatment. In certain embodiments, the patient has a reduction in the loss of ambulation for at least 24 weeks immediately after commencing treatment as compared to a placebo control.

In certain embodiments, treatment is measured by assaying the serum of the patient for biomarkers of inflammation. In certain embodiments, the treatment results in a reduction in the levels of one or more, or a combination of biomarkers of inflammation. For example, in certain embodiments, the biomarkers of inflammation are one or more or a combination of the following: cytokines (such as IL-1, IL-6, TNF-α), C-reactive protein (CRP), leptin, adiponectin, and creatine kinase (CK). In certain embodiments, treatment lowers levels of the p65 subunit of NF-κB compared to treatment with a placebo. Biomarkers of inflammation may be assayed by methods known in the art, for example, as described in Cruz-Guzman et al. (2015) BIOMED RESEARCH INTERNATIONAL 891972.

In certain embodiments, treatment maintains or increases a stable walking distance in a patient, as measured by, for example, the 6 Minute Walk Test (6MWT), described, for example, in McDonald et al. (2010) MUSCLE NERVE 42:966-74. A change in the 6 Minute Walk Distance (6MWD) may be expressed as an absolute value, a percentage change or a change in the %-predicted value. In certain embodiments, treatment maintains or improves a stable walking distance in a 6MWT from a 20% deficit in the subject relative to a healthy peer. The performance of a DMD patient in the 6MWT relative to the typical performance of a healthy peer can be determined by calculating a %-predicted value. For example, the %-predicted 6MWD may be calculated using the following equation for males: 196.72+(39.81×age)−(1.36×age²)+(132.28×height in meters). For females, the %-predicted 6MWD may be calculated using the following equation: 188.61+(51.50×age)−(1.86×age²)+(86.10×height in meters) (Henricson et al. (2013) PLOS CURR 5). In certain embodiments, treatment with an antisense oligonucleotide increases the stable walking distance in the patient from baseline to greater than 3, 5, 6, 7, 8, 9, 10, 15, 20, 25, 30 or 50 meters (including all integers in between).

Loss of muscle function in patients with DMD may occur against the background of normal childhood growth and development. Indeed, younger children with DMD may show an increase in distance walked during 6MWT over the course of about 1 year despite progressive muscular impairment. In certain embodiments, the 6MWD from patients with DMD is compared to typically developing control subjects and to existing normative data from age and sex matched subjects. In certain embodiments, normal growth and development can be accounted for using an age and height based equation fitted to normative data. Such an equation can be used to convert 6MWD to a percent-predicted (%-predicted) value in subjects with DMD. In certain embodiments, analysis of %-predicted 6MWD data represents a method to account for normal growth and development, and may show that gains in function at early ages (e.g., less than or equal to age 7) represent stable rather than improving abilities in patients with DMD (Henricson et al. (2013) supra).

Additional exemplary muscular dystrophies include Becker's muscular dystrophy (BMD), congenital muscular dystrophy, distal muscular dystrophy, Emery-Dreifuss muscular dystrophy, facioscapulohumeral muscular dystrophy, limb-girdle muscular dystrophy, myotonic muscular dystrophy and oculopharyngeal muscular dystrophy.

In certain embodiments, the method reduces inflammation in quadriceps muscle by at least 20%, and/or reduces fibrosis in quadriceps muscle by at least 20%.

The methods and compositions of the invention may also be used to treat an inflammatory disease in a subject. The inflammation can be associated with an inflammatory disease or a disease where inflammation contributes to the disease. Inflammatory diseases can arise where there is an inflammation of the body tissue. These include local inflammatory responses and systemic inflammation. Examples of such diseases include, but are not limited to: organ transplant rejection; reoxygenation injury resulting from organ transplantation (Grupp et al. (1999) J. MOL. CELL. CARDIOL. 31: 297-303) including, but not limited to, transplantation of the following organs: heart, lung, liver and kidney; chronic inflammatory diseases of the joints, including arthritis, rheumatoid arthritis, osteoarthritis and bone diseases associated with increased bone resorption; inflammatory bowel diseases such as ileitis, ulcerative colitis, Barrett's syndrome, and Crohn's disease; inflammatory lung diseases such as asthma, adult respiratory distress syndrome, chronic obstructive airway disease, and cystic fibrosis; inflammatory diseases of the eye including corneal dystrophy, trachoma, onchocerciasis, uveitis, sympathetic ophthalmitis and endophthalmitis; chronic inflammatory diseases of the gum, including gingivitis and periodontitis; inflammatory diseases of the kidney including uremic complications, glomerulonephritis and nephrosis; inflammatory diseases of the skin including sclerodermatitis, psoriasis and eczema; inflammatory diseases of the central nervous system, including chronic demyelinating diseases of the nervous system, multiple sclerosis, AIDS-related neurodegeneration and Alzheimer's disease, infectious meningitis, encephalomyelitis, Parkinson's disease, Huntington's disease, amyotrophic lateral sclerosis and viral or autoimmune encephalitis. Metabolic disease such as type II diabetes mellitus; the prevention of type I diabetes; dyslipedemia; hypertriglyceridemia; diabetic complications, including, but not limited to glaucoma, retinopathy, macula edema, nephropathy, such as microalbuminuria and progressive diabetic nephropathy, polyneuropathy, diabetic neuropathy, atherosclerotic coronary arterial disease, peripheral arterial disease, nonketotic hyperglycemichyperosmolar coma, mononeuropathies, autonomic neuropathy, joint problems, and a skin or mucous membrane complication, such as an infection, a shin spot, a candidal infection or necrobiosis lipoidica diabeticorum; immune-complex vasculitis, systemic lupus erythematosus; inflammatory diseases of the heart such as cardiomyopathy, ischemic heart disease hypercholesterolemia, and atherosclerosis; as well as various other diseases that can have significant inflammatory components, including preeclampsia; chronic liver failure, brain and spinal cord trauma, and cancer. The inflammatory disease can also be a systemic inflammation of the body, exemplified by gram-positive or gram-negative shock, hemorrhagic or anaphylactic shock, or shock induced by cancer chemotherapy in response to proinflammatory cytokines, e.g., shock associated with proinflammatory cytokines. Such shock can be induced, e.g., by a chemotherapeutic agent that is administered as a treatment for cancer. Other disorders include depression, obesity, allergic diseases, acute cardiovascular events, arrhythmia, prevention of sudden death, inflammatory myopathies such as dermatomositis, inclusion body myositis, and polymyositis, cancer cachexia, and inflammation that results from surgery and trauma.

In certain embodiments, a method or composition of the invention is administered in combination with corticosteroid. Corticosteroids are a class of chemicals that includes steroid hormones naturally produced in the adrenal cortex of vertebrates and analogues of these hormones that are synthesized in laboratories. Corticosteroids are involved in a wide range of physiological processes, including stress response, immune response, and regulation of inflammation, carbohydrate metabolism, protein catabolism, blood electrolyte levels, and behavior. Exemplary corticosteroids include betamethasone, budesonide, cortisone, dexamethasone, hydrocortisone, methylprednisolone, prednisolone, prednisone, or deflazacort. In certain embodiments, the corticosteroid is administered prior to, in conjunction with, or subsequent to a method or composition of the invention.

In certain embodiments, a method or composition of the invention is administered in combination with an exon skipping agent, for example, Exondys 51® (eteplirsen; see U.S. Pat. Nos. 7,807,816, 7,960,541, 8,486,907, 9,416,361, and 9,506,058), which has been approved by the United States Food and Drug Administration for the treatment of DMD in patients who have a confirmed mutation of the DMD gene that is amenable to exon 51 skipping. It is contemplated that other exon skipping agents, for example, exon 45 skipping agents, such as SRP-4045 (see, U.S. Pat. Nos. 8,524,880, 9,447,415 and 9228,187), other exon skipping agents such as drisapersen, and exon 53 skipping agents, such as SRP-4053 (see, U.S. Pat. Nos. 8,455,636, 9,024,007, and 9,416,361) may be administered in combination with an active ingredient, e.g., edasalonexent, described herein.

In certain embodiments, the subject is a human. In certain embodiments, the subject is seven years of age or older. In certain embodiments, the subject is between about 6 months and about 4 years of age. In certain embodiments, the subject is between about 4 years of age and 7 years of age.

### IV. Dosing/Administration

The dosage regimen utilizing the active ingredient, e.g., edasalonexent, is selected in accordance with a variety of factors including type, species, age, weight, sex and medical condition of the patient; the severity of the condition to be treated; the route of administration; the renal or hepatic function of the patient; and the particular compound employed.

In certain embodiments, a dose of the fatty acid acetylated salicylate, e.g., edasalonexent, administered to the patient comprises between about 10 mg/kg and about 500 mg/kg (e.g., about 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450, or 500 mg/kg). In certain embodiments, a dose of the active ingredient, e.g., edasalonexent, administered to the patient comprises from about 10 mg/kg to about 100 mg/kg, from about 10 mg/kg to about 75 mg/kg, from about 10 mg/kg to about 50 mg/kg, from about 10 mg/kg to about 25 mg/kg, from about 25 mg/kg to about 100 mg/kg, from about 25 mg/kg to about 75 mg/kg, from about 25 mg/kg to about 50 mg/kg, from about 50 mg/kg to about 100 mg/kg, from about 50 mg/kg to about 75 mg/kg, from about 75 mg/kg to about 100 mg/kg, or from about 90 mg/kg to about 110 mg/kg. In certain embodiments, a dose of the active ingredient, e.g., edasalonexent, administered to the patient comprises between about 25 mg/kg and 50 mg/kg, e.g., about 33 mg/kg or between about 50 mg and about 100 mg/kg, e.g., about 67 mg/kg. Alternatively, dosages may be given in absolute terms, for example, 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 250 mg, 300 mg, 350mg, 400 mg, 450 mg, 500 mg, 550 mg, 600 mg, 650 mg, 700 mg, 750 mg, 800 mg, 850 mg, 900 mg, 950 mg, 1000 mg, 1500 mg, 2000 mg, 2500 mg, 3000 mg, 3500 mg, 4000 mg, 4500 mg, 5000 mg, 5500 mg, 6000 mg, 6500 mg, 7000 mg, 7500 mg, 8000 mg, 8500 mg, 9000 mg, 9500 mg, or 10,000mg. In certain embodiments, the dose is administered as one or more dosage forms containing 100 mg, 250 mg, 500 mg or 1000 mg of the active ingredient, e.g., edasalonexent. For example, in one embodiment, the dosage form contains 100 mg of edasalonexent. In another embodiment, the dosage form contains 250 mg of edasalonexent. In certain embodiments, the total daily dose is about 1500 mg to about 3000 mg or about 2000 mg to about 3000 mg. In certain embodiments, the total daily dose is delivered in three divided doses ranging from about 500 mg to about 1000 mg or from about 670 mg to about 1000 mg.

In certain embodiments, a dose of the active ingredient, e.g., edasalonexent, is administered to the patient once per day. In certain embodiments, a dose of the active ingredient, e.g., edasalonexent, is administered to the patient more than once per day. For example, in certain embodiments, a dose of the active ingredient, e.g., edasalonexent, is administered to the patient, e.g., twice per day, three times per day, or four times per day.

In certain embodiments wherein more than one dose of the active ingredient, e.g., edasalonexent, is administered to the patient per day, each dose comprises an equal amount of the fatty acid acetylated salicylate. In other embodiments, one or more doses may comprise a different amount of the active ingredient, e.g., edasalonexent, than another dose.

In certain embodiments, a dosing regimen comprises three doses per day. In certain embodiments, the three doses comprise equal amounts of the compound, e.g., each dose comprises from about 25 mg/kg to about 50 mg/kg of the compound, e.g., each dose comprises about 33 mg/kg of the compound. In certain embodiments, the first dose and the second dose comprise a smaller amount of the active ingredient, e.g., edasalonexent, than the third dose, e.g., the first dose and the second dose comprise about half the amount of the active ingredient, e.g., edasalonexent, as the third dose. For example, in certain embodiments, the first dose and the second dose comprise from about 25 mg/kg to about 50 mg/kg of the active ingredient, e.g., edasalonexent, and the third dose comprises from about 50 mg/kg to about 100 mg/kg of the active ingredient, e.g., edasalonexent, e.g., the first dose and the second dose comprise about 33 mg/kg of active ingredient, e.g., edasalonexent, and the third dose comprises about 67 mg/kg of the active ingredient, e.g., edasalonexent. In certain embodiments, of the three daily doses, at least one dose is different in amount than one other dose. In certain embodiments, of the three daily doses, two are the same and one is different. For example, in certain embodiments, each dose comprises from about 20 mg/kg to about 40 mg/kg. For example, two doses comprise about 37.5 mg/kg, while the remaining dose comprises about 25 mg/kg. In certain embodiments, all three doses are different. For example, in certain embodiments, each dose comprises from about 20 mg/kg to about 40 mg/kg.

In certain embodiments, the first dose is administered in the morning, the second dose is administered at mid-day, and the third dose is administered in the evening. In certain embodiments, each dose is administered with food, e.g., at the time of a meal. For example, in certain embodiments, the first dose is administered at the time of breakfast, the second dose is administered at the time of lunch, and the third dose is administered at the time of dinner. When the dose is taken with food, the food content may be adjusted to facilitate absorption of the active compound. For example, the dose may be taken with high-fat meals. For example, in one embodiment, the dose is taken with a meal containing at least 1 g, at least 2 g, at least 3 g, at least 4g, at least 5 g, at least 6 g, at least 7 g, at least 8 g, at least 9 g, at least 10 g, at least 11 g, at least 12 g, at least 13 g, at least 14 g, at least 15 g, at least 16 g, at least 17 g, at least 18 g, at least 19 g, or at least 20 g of fat. In one particular embodiment, the dose is taken with a meal containing at least 8 g of fat. Under certain circumstances, the same doses are administered with breakfast and dinner, whereas a smaller dose is administered with lunch. Under certain circumstances, where three daily doses are administered and the doses are not equal, the smallest dose is taken at lunch time. In certain embodiments, where three daily doses are administered and the doses are not equal, the largest dose is taken at dinner time.

In certain embodiments, the total daily dosage of the active ingredient, e.g., edasalonexent, administered to the patient comprises between about 20 mg/kg and about 1000 mg/kg (e.g., about 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, or 1000 mg/kg). In certain embodiments, the total daily dosage of the active ingredient, e.g., edasalonexent, administered to the patient comprises from about 100 mg/kg to about 200 mg/kg, about 100 mg/kg to about 175 mg/kg, about 100 mg/kg to about 150 mg/kg, about 100 mg/kg to about 125 mg/kg, about 125 mg/kg to about 200 mg/kg, about 125 mg/kg to about 175 mg/kg, about 125 mg/kg to about 150 mg/kg, about 150 mg/kg to about 200 mg/kg, about 150 mg/kg to about 175 mg/kg, or about 175 mg/kg to about 200 mg/kg. In certain embodiments, the total daily dosage of the active ingredient, e.g., edasalonexent, comprises between about 100 mg/kg and 200 mg/kg, e.g., about 100 mg/kg or about 133 mg/kg.

In certain embodiments, the total daily dosage of the active ingredient, e.g., edasalonexent, administered to the patient comprises from about 90 mg/kg to about 110 mg/kg. In certain embodiments, the total daily dosage of the active ingredient, e.g., edasalonexent, administered to the patient comprises 100 mg/kg. In certain embodiments, the total daily dosage of the active ingredient, e.g., edasalonexent, administered to the patient comprises 100 mg/kg±5% of the total daily dose. In certain embodiments, the total daily dosage of the active ingredient, e.g., edasalonexent, administered to the patient comprises 100 mg/kg±10% of the total daily dose. In certain embodiments, the total daily dosage of the active ingredient, e.g., edasalonexent, administered to the patient comprises 100 mg/kg±15% of the total daily dose. In certain embodiments, the total daily dosage of the active ingredient, e.g., edasalonexent, administered to the patient comprises 100 mg/kg±20% of the total daily dose.

In certain embodiments, the total daily dosage of the active ingredient, e.g., edasalonexent, is administered to the patient in three divided doses wherein each of the three doses is either a multiple of 250 mg (e.g., 250 mg, 500 mg, 750 mg, 1000 mg, 1250 mg, 1500 mg, 1750 mg, or 2000 mg, etc.) or a multiple of 100 mg (e.g., 100 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, or 2000 mg, etc.). In certain embodiments, the dose is administered as one or more dosage forms containing 100 mg or 250 mg of the active ingredient, e.g., edasalonexent. In certain embodiments, the three divided doses are the same and are multiples of 250 mg or 100 mg. In certain embodiments, of the three divided doses, two doses are equal and one is different and the doses are multiples of 250 mg or 100 mg. In certain embodiments, the three divided doses are each different, but the doses are multiples of 250 mg or 100 mg. In certain embodiments, the three divided doses are the same, are multiples of 250 mg or 100 mg and provide a total daily dose of 100 mg/kg±10%. In certain embodiments, of the three divided doses, two doses are equal and one is different, the doses are multiples of 250 mg or 100 mg, and provide a total daily dose of 100 mg/kg±10%. In certain embodiments, the three divided doses are each different, but the doses are multiples of 250 mg or 100 mg and provide a total daily dose of 100 mg/kg±10%. In certain embodiments, the three divided doses are the same, are multiples of 250 mg or 100 mg and provide a total daily dose of 100 mg/kg±15%. In certain embodiments, of the three divided doses, two doses are equal and one is different, the doses are multiples of 250 mg or 100 mg, and provide a total daily dose of 100 mg/kg±15%. In certain embodiments, the three divided doses are each different, but the doses are multiples of 250 mg or 100 mg and provide a total daily dose of 100 mg/kg±15%. In certain embodiments, the three divided doses are the same, are multiples of 250 mg or 100 mg and provide a total daily dose of 100 mg/kg±20% . In certain embodiments, of the three divided doses, two doses are equal and one is different, the doses are multiples of 250 mg or 100 mg, and provide a total daily dose of 100 mg/kg±20%. In certain embodiments, the three divided doses are each different, but the doses are multiples of 250 mg or 100 mg and provide a total daily dose of 100 mg/kg±20%. In certain embodiment, the total daily dosage of the active ingredient, e.g., edasalonexent, does not exceed 6000 mg.

The methods and pharmaceutical compositions described herein may be chronically administered to the patient for the treatment of muscular dystrophy. For example, the methods or pharmaceutical compositions may be administered for a period of time of at least several weeks or months or years (e.g., for at least 2 weeks, 4 weeks, 6 weeks, 8 weeks, 10 weeks, 12 weeks, 24 weeks, 36 weeks, 48 weeks, 72 weeks, 96 weeks, 120 weeks, 144 weeks, 168 weeks, 180 weeks, 192 weeks, 216 weeks, or at least 240 weeks).

As noted above, the formulations or preparations disclosed herein may be given orally, parenterally, systemically, topically, rectally or intramuscular administration. They are typically given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. In certain embodiments, the active ingredient, e.g., edasalonexent, can be administered in intranasal form via topical use of suitable intranasal vehicles, or via transdermal routes, using those forms of transdermal skin patches well known to those of ordinary skill in that art. In certain other embodiments, dosage form is administered orally. In certain embodiments, the dosage form is administered orally as a capsule or tablet.

In certain embodiments and as shown in Example 4, treatment of children suffering from Duchenne Muscular Dystrophy with 100 mg/kg/day edasalonexent in three equal doses (about 33 mg/kg) provides a clinically meaningful slowing of disease progression, e.g., as demonstrated by 10 meter walk/run, 4 stair climb, and time to stand times, and stabilizes the North Star Ambulatory Assessment (NSAA) score over at least a 60 week treatment period as compared to an off-treatment control period.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby.

### Example 1: Evaluation of Edasalonexent Dosing Regimen Pharmacokinetics in C57BL/6 Mice

C57BL/6 mice were administered edasalonexent in their diet and/or by oral gavage. Pharmacokinetic (PK) modeling was used to identify suitable dosing amounts and timing in relation to the doses of 67 mg/kg/day and 100 mg/kg/day that have been tested in edasalonexent human clinical trials. Specifically, mice were administered 1.5% edasalonexent in their diet, 450 mg/kg/day delivered orally as single daily dose, 450 mg/kg/day delivered orally as three equally divided doses, or 600 mg/kg/day delivered orally as two 150 mg/kg/doses and one 300 mg/kg/dose. For dosing regimens with three daily doses, doses were delivered at 7:00 am, noon and 5:00 pm.

The dosing regimens for each group are depicted in TABLE 1. Each group included 12 mice.

TABLE 1

| Group | Diet | Oral Treatment |
|---|---|---|
| 1 | Edasalonexent | |
| | Diet (1.5% w/w) | |
| 2 | Control Diet | Edasalonexent QD |
| | | (450 mg/kg/dose) |
| 3 | Control Diet | Edasalonexent TID |
| | | (150 mg/kg/dose = 450 mg/kg/day) |
| 4 | Control Diet | Edasalonexent TID |
| | | (150/150/300 mg/kg/dose = 600 mg/kg/day) |

For animals administered edasalonexent in their diet, edasalonexent concentration was measured by LC/MS/MS every 4 hours for 24 hours, beginning on the seventh day of dosing. For animals administered edasalonexent orally, edasalonexent concentration was measured by LC/MS/MS at 0.25, 0.5, 1, 2, 4, 6 and 24 hours after dosing, beginning on the third day of dosing.

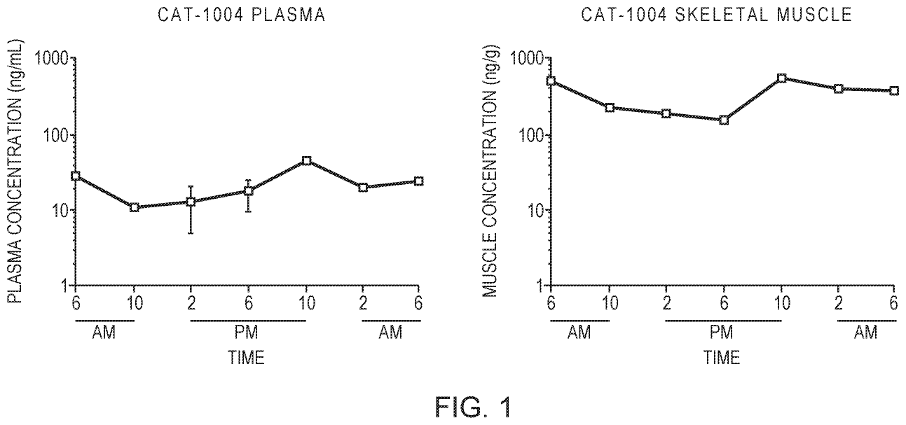

Edasalonexent concentration in the indicated tissue for group 1, receiving a dose of 1.5% edasalonexent in the diet, is depicted in FIG. 1, and PK parameters for this group are shown in TABLE 2.

TABLE 2

| | Cₘₑₐₙ | Cₘₐₓ | Cₘᵢₙ | AUC |
|---|---|---|---|---|
| Tissue | (ng/mL) | (ng/mL) | (ng/mL) | (hr*ng/mL) |
| Plasma | 22.4 | 44.6 | 10.7 | 524.0 |
| Gastrocnemius | 332.8 | 532.3 | 149.4 | 7615 |

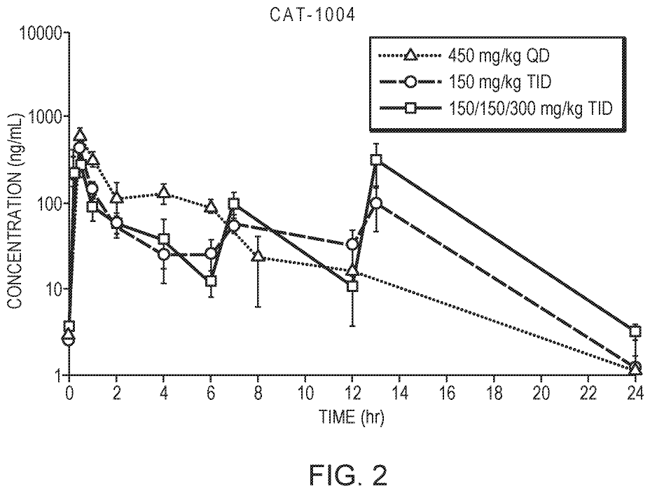

Plasma edasalonexent concentrations for treatment group 2 (receiving 450 mg/kg/day delivered orally as single daily dose), group 3 (receiving 450 mg/kg/day delivered orally as three equally divided doses), and group 4 (receiving 600 mg/kg/day delivered orally as two 150 mg/kg/doses and one 300 mg/kg/dose) are depicted in FIG. 2, and PK parameters for these groups are shown in TABLE 3. Group 2 displayed intermittent high exposure to edasalonexent and an AUC of 1316 hr*ng/mL, ˜2.5-fold higher than that observed for group 1.

TABLE 3

| | Tₘₐₓ | Cₘₐₓ | AUC_{0-24 h, day 3} |
|---|---|---|---|
| Group | (h) | (ng/mL) (±SE) | (hr*ng/mL) (±SE) |
| 2 | 0.5 | 604 ± 83.3 | 1316 ± 126 |
| 3 | 0.5 | 452 ± 47.6 | 1365 ± 211 |
| 4 | 0.5 | 278 ± 46.3 | 2269 ± 587 |

### Example 2: Evaluation of Edasalonexent Dosing Regimens in mdx Mice

Young mdx mice were treated for four weeks with varying dosing regimens of edasalonexent. The mdx mouse is a useful and generally accepted animal model for studying Duchenne muscular dystrophy (DMD) (Mann et al. (2001) PROC. NATL. ACAD. S_{CI.} 98(1):42-7). mdx mice are deficient in expression of full-length dystrophin due to a genetic mutation within the dystrophin gene.

Mice were administered edasalonexent in their diet, and/or by oral gavage. Specifically, mice were administered 1% edasalonexent in their diet, 450 mg/kg/day delivered orally as single daily dose, 450 mg/kg/day delivered orally as three equally divided doses, and/or 600 mg/kg/day delivered orally as two 150 mg/kg/doses and one 300 mg/kg/dose. For dosing regimens with three daily doses, doses were delivered at 7:00 am, noon and 5:00 pm.

The dosing regimens for each treatment group are depicted in TABLE 4. Each treatment group included 10-11 mice.

TABLE 4

| Group | Diet | Oral Treatment |
|---|---|---|
| 1 | Control Diet | |
| 2 | Edasalonexent | |
| | Diet (1% w/w) | |
| 3 | Control Diet | Vehicle - TID |
| 4 | Control Diet | Edasalonexent QD |
| | | (450 mg/kg/dose) |
| 5 | Edasalonexent | Edasalonexent QD |
| | Diet (1% w/w) | (450 mg/kg/dose) |
| 6 | Control Diet | Edasalonexent TID |
| | | (150 mg/kg/dose = 450 mg/kg/day) |
| 7 | Control Diet | Edasalonexent TID |
| | | (150/150/300 mg/kg/dose = 600 mg/kg/day) |

Animals were treated for four weeks, sacrificed, and tissues were collected for histological analysis. For assessment of inflammation and fibrosis, quadriceps muscles were processed for hematoxylin and eosin (H&E) and picrosirius red (collagen) histochemical staining. From each animal, 2-4 complete transverse sections were digitally analyzed using ImageJ, with the hematoxylin-stained nuclear area determined as a measure of inflammatory infiltration, and the picrosirius red-stained area determined as a measure of fibrosis.

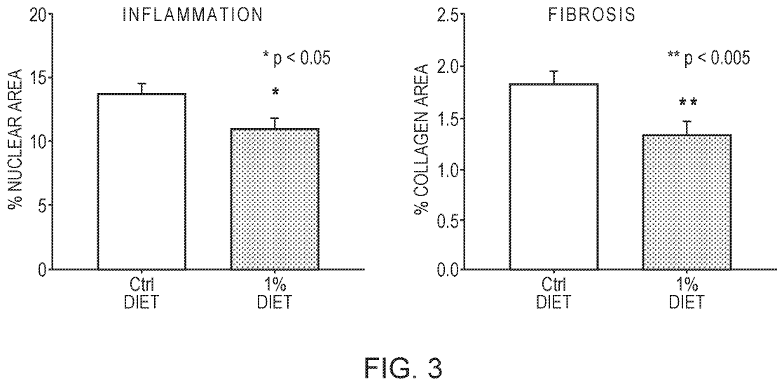

Inflammation and fibrosis in treatment group 2, as measured by histochemical staining, are depicted in FIG. 3. Extending previous studies in older mice, 1% edasalonexent in the diet reduced inflammation and fibrosis in young mdx mice. Combined with PK data from Example 1, these results s how that administration of edasalonexent in diet resulted in constant, low exposure and reduced inflammation and fibrosis.

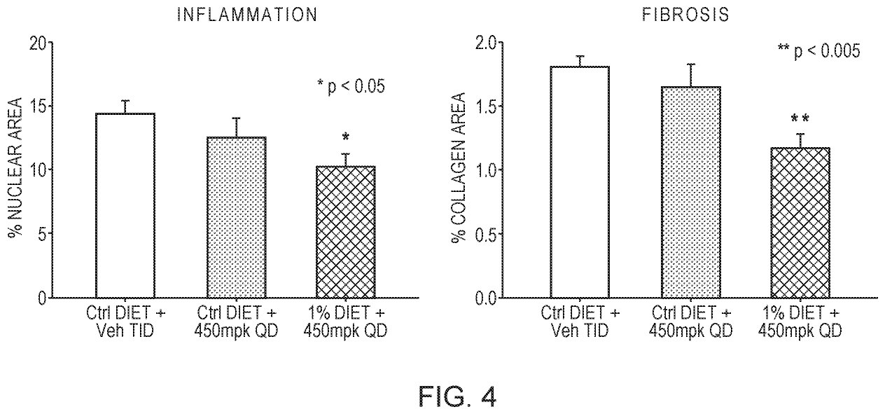

Inflammation and fibrosis for treatment groups 3, 4, and 5 are depicted in FIG. 4. The results show that treatment group 4 did not cause a significant reduction of inflammation or fibrosis. Therefore, administration of edasalonexent in the diet (group 2; FIG. 3) was effective, while administration of edasalonexent with a single daily 450 mg/kg/dose (group 4; FIG. 4) was not effective, despite a ˜2.5-fold higher AUC for the single daily 450 mg/kg/dose, as observed in C57BL/6 mice in Example 1. These results show that intermittent high exposure to edasalonexent by oral gavage is not efficacious.

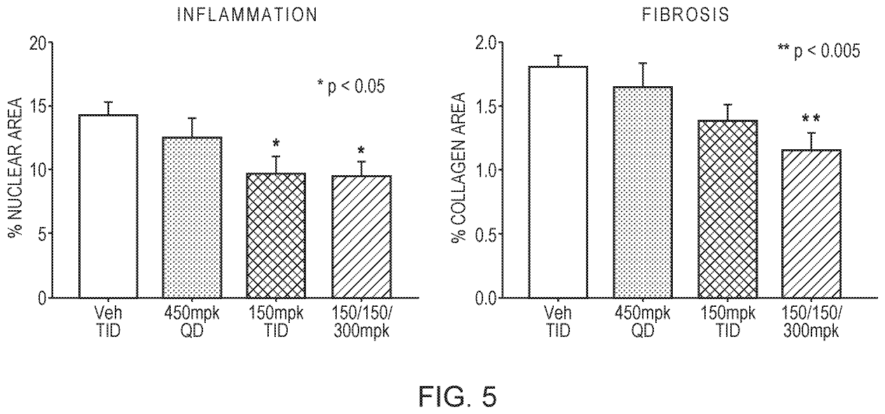

Inflammation and fibrosis for treatment groups 6 and 7 are depicted in FIG. 5. The results show that dosing frequency drives efficacy, and increasing temporal coverage by dosing three times a day improved therapeutic outcomes. Administration of edasalonexent with a single daily 450 mg/kg dose (group 4; FIG. 4) was not effective, but administration of three daily 150 mg/kg doses, for the same total daily dose (group 6; FIG. 5), was effective, despite the fact that the two treatments result in similar maximum (Cₘₐₓ) and minimum concentrations, and total drug exposure (AUC) in PK studies in C57BL/6 mice. Doubling the last daily dose (group 7) further drove efficacy. It is believed the last dose provided additional exposure during the long overnight trough period.

Together, these results suggest that maximum time over a certain threshold drug concentration, rather than Cₘₐₓ or drug total exposure, primarily drives efficacy for edasalonexent.

### Example 3: Modeling of Human Edasalonexent Dosing Regimens

Edasalonexent was shown to be safe and well-tolerated, and inhibited activated NF-κB pathways in a phase 1 clinical program that included three placebo-controlled studies in adults (Donovan et al. (2017) J. CLIN. PHARMACOL. 57(5): 627-639). Edasalonexent has also shown positive treatment effects in boys affected by DMD enrolled at age 4-7 in the MoveDMD® phase 2 trial (NCT02439216) and its open-label extension.

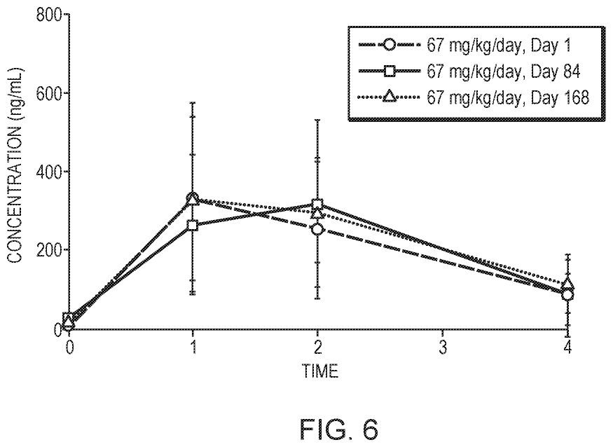

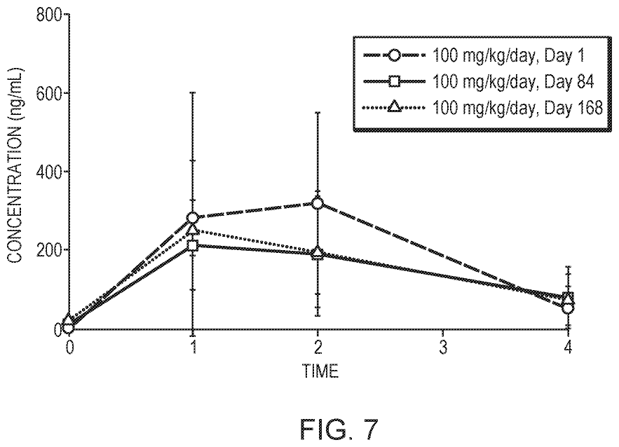

FIG. 6 and FIG. 7 show edasalonexent plasma concentration for subjects from the MoveDMD® phase 2 trial. Subjects received either two 33 mg/kg doses per day (for a total daily dose of 67 mg/kg/day) or three 33 mg/kg doses per day (for a total daily dose of 100 mg/kg/day). Concentration measurements were taken after the first daily 33 mg/kg dose for either group.

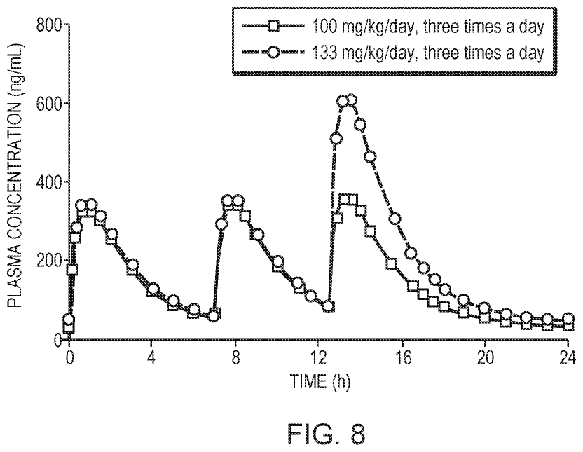

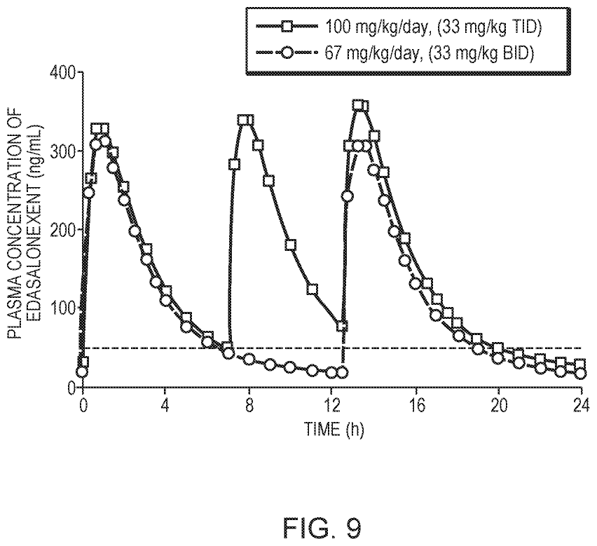

A population PK model was developed using data from the phase 1 and phase 2 edasalonexent clinical trials. The plasma concentration and PK parameters were modeled for three dosing regimens: two 33 mg/kg doses per day (for a total daily dose of 87 mg/kg), three 33 mg/kg doses per day (for a total daily dose of 100 mg/kg/day), or two 33 mg/kg doses and one 67 mg/kg dose per day (for a total daily dose of 133 mg/kg/day). The results are shown in FIGS. 8 and 9 and TABLE 5.

TABLE 5

| Cₘₐₓ (ng/mL) | | Cₘᵢₙ (ng/mL) | | AUC0-24 (h*ng/mL) | |
|---|---|---|---|---|---|
| 133 mg/kg | 100 mg/kg | 133 mg/kg | 100 mg/kg | 133 mg/kg | 100 mg/kg |
| 651 | 390 | 38.2 | 27.5 | 4700 | 3670 |
| 646 | 450 | 35.8 | 25.9 | 4690 | 3670 |
| 661 | 385 | 41.4 | 29.6 | 4700 | 3670 |
| 640 | 413 | 40.5 | 28.6 | 4700 | 3670 |
| 679 | 404 | 36.7 | 26.8 | 4690 | 3670 |

The results show that the Cₘᵢₙ for the 100 mg/kg/day dosing regimen (33 mg/kg, TID) is in the range of efficacious levels in the mdx mouse model, and doubling the evening dose to give the 133 mg/kg/day dosing regimen increases time over threshold. Further, as shown in FIG. 9, the 100 mg/kg (33 mg/kg TID) dose results in a substantial increase in time over threshold compared to the 67 mg/kg (33 mg/kg BID) dose.

These results, combined with those from Examples 1 and 2 herein, provide preclinical support to evaluate an equally divided 100 mg/kg/day clinical dose (three 33 mg/kg doses) as well as an unequally divided 133 mg/kg/day clinical dose (two 33 mg/kg doses and one 67 mg/kg dose per day), as the data suggest these doses may provide sufficient time over threshold to drive efficacy in the treatment of muscular dystrophy, e.g., DMD, in humans.

### Example 4: Evaluation of Edasalonexent in Human DMD Patients

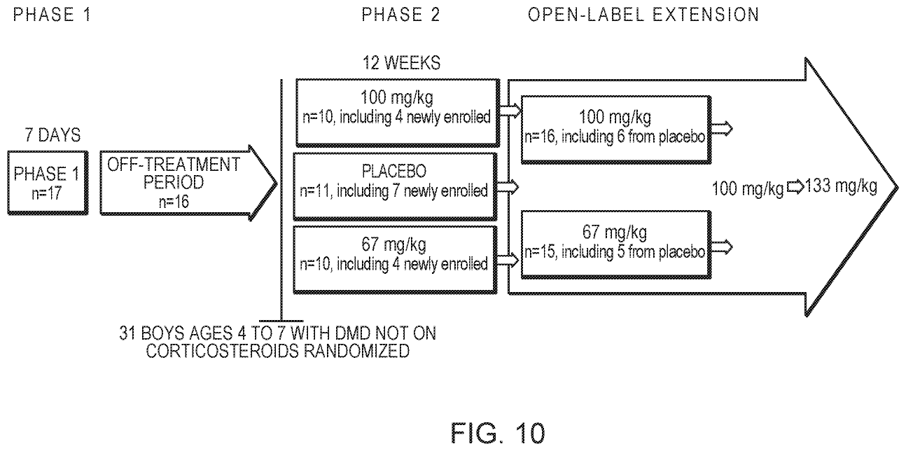

The MoveDMD trial (see FIG. 10) was designed to evaluate efficacy, safety/tolerability, pharmacokinetics (PK), pharmacodynamics (PD), and dose response of edasalonexent. The first phase portion of the MoveDMD trial was a 1-week study to evaluate the safety, PK, and PD at 33 and 67 mg/kg/day, given in 2 divided doses, and 100 mg/kg/day given in 3 divided doses.

The second phase and the open-label extension (OLE) portions of the MoveDMD trial enrolled boys ages 4 through 7 with DMD. Phase 2 was a 12-week placebo-controlled study with two cohorts given 33 mg/kg twice daily (BID; 67 mg/kg/day; n=10) or 33 mg/kg three times daily (TID; 100 mg/kg/day; n=10), and a placebo cohort (n=10). Patients in the OLE continued with either dose regimen of edasalonexent.

The dose schedule for the second phase of the MoveDMD trial was selected based on nonclinical and clinical data including (1) the exposure/efficacy relationship observed in animal models; (2) the Phase 1 safety, tolerability, and PD in pediatric DMD patients; and (3) human PK.

Edasalonexent administered 33 mg/kg three times daily (TID) (100 mg/kg/day) demonstrated a preservation of muscle function and slowing of DMD disease progression through 60 weeks as compared to the rates of change during the control period prior to edasalonexent treatment. It was also observed that this 100 mg/kg/day dosing regimen was equally effective as the dosing regimen containing 133 mg/kg/day (33 mg/kg at breakfast, 33 mg/kg at lunch, and 67 mg/kg at dinner) with fewer side effects.

4.A Systemic Exposure in DMD Patients Achieves Levels at which NF-κB Inhibition was Observed in Adults

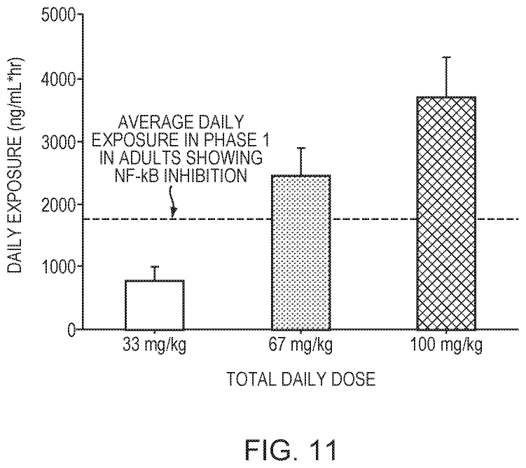

In an adult study, changes in expression of NF-κB driven genes were observed in whole blood at a dose of approximately 100 mg/kg/day. In the MoveDMD study, when doses of 33 mg/kg were given twice daily (total dose of 67 mg/kg) or three times daily (total dose of 100 mg/kg), systemic exposures reached levels at which NF-κB was observed in the adult study. FIG. 11 shows the daily exposure levels (ng/ml*hr) for boys enrolled in the Phase II study as compared to the average daily exposure in adults showing NF-κB inhibition. As shown, the 67 mg/kg/day and 100 mg/kg/day doses provide daily exposure levels comparable to the those levels shown in the adult study to be effective in inhibiting NF-κ B.

4.B Sustained Edasalonexent Exposure in DMD Patients Correlates with NF-κB Inhibition

To determine the effects of a given edasalonexent dosing regimen on NF-κB inhibition, the expression levels of a number of NF-κB regulated and inflammation-related gene transcripts were measured in whole blood samples from DMD patients.

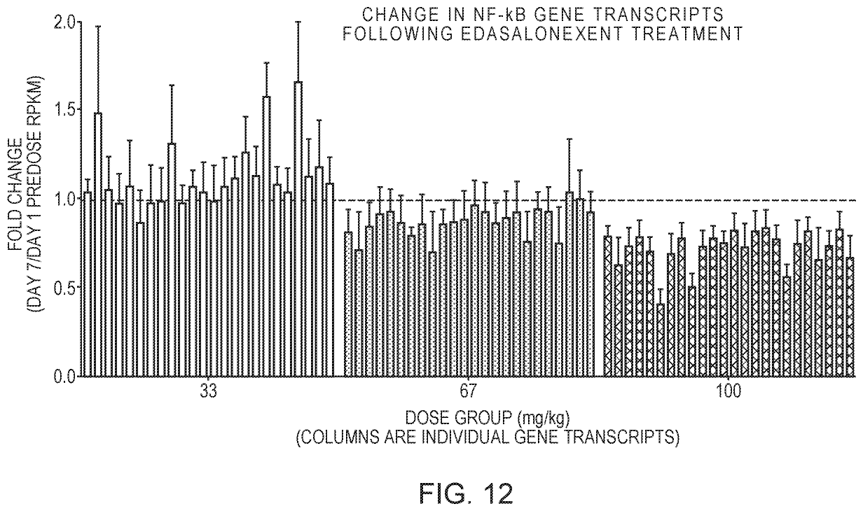

NF-kB regulated and inflammation-related genes were chosen from the Broad Institute's HALLMARK TNF/NF-kB gene set (Liberzon, et. al., (2015), Cell Systems, 1(6):417-425). Blood was drawn from DMD boys before and one-week after edasalonexent treatment, and collected into PAXgene RNA tubes (Qiagen). RNA was extracted and sequenced to directly measure the abundance of transcripts in whole blood for both time points in each patient, and the abundance of transcripts one-week post treatment was compared to pre-treatment values to determine the relative change in each of the selected transcripts. This ratio for each gene across all patients within a dose cohort was averaged and is shown in FIG. 12 by columns as average +/−SEM. A value less than 1 indicates a decrease in the transcripts after one-week treatment with edasalonexent.

As shown in FIG. 12, there was a statistically significant decrease in all 24 transcripts measured in the group receiving the 100 mg/kg/day dose, whereas the decrease at the 67 mg/kg/day dose was not so pronounced. At the 33 mg/kg/day dose, decreases were generally not noted.

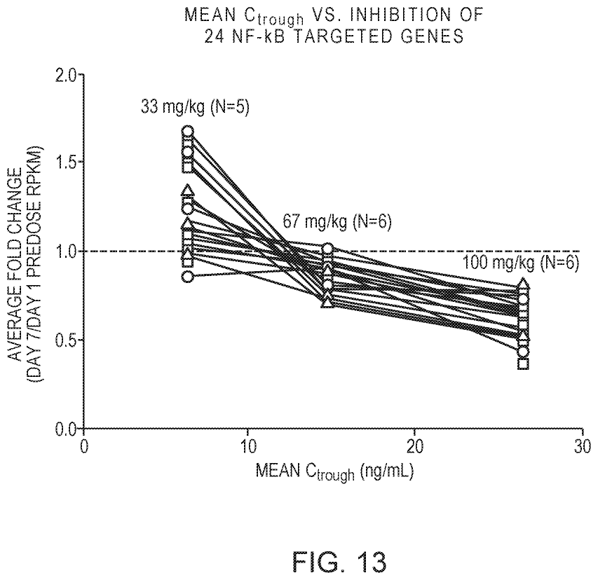

The ratios of all 24 transcripts within a patient were also averaged, and are shown in FIG. 13 using symbols which represent this average from each individual patient. This average was graphed against the average edasalonexent C_{trough} values within each dose cohort (shown on the x-axis in ng/ml).

FIG. 13 shows the average fold change in gene transcripts versus the mean C_{trough} levels (ng/mL) for each of the 33 mg/kg/day, 67 mg/kg/day, and 100 mg/kg/day edasalonexent doses. As shown in FIG. 13, a higher mean C_{trough} level correlates with the highest decrease in NF-κB and inflammation related gene transcriptions. In particular, the 100 mg/kg/day dose had the highest C_{trough} level and highest decrease in relevant gene transcripts as compared to the 33 mg/kg/day and 67 mg/kg/day doses, showing an inverse correlation in the abundance of transcripts with the mean C_{trough} levels after one-week of edasalonexent treatment. Accordingly, the results show that, of these three dosing regimens, 100 mg/kg/day (33 mg/kg three times daily) was the most effective in reducing NF-κB regulated and inflammation-related gene transcripts in DMD patients, and suggest that C_{trough} is a driver edasalonexent's efficacy.

4. C Edasalonexent Treatment Stabilizes NSAA and Other Functional Measures in Boys with DMD

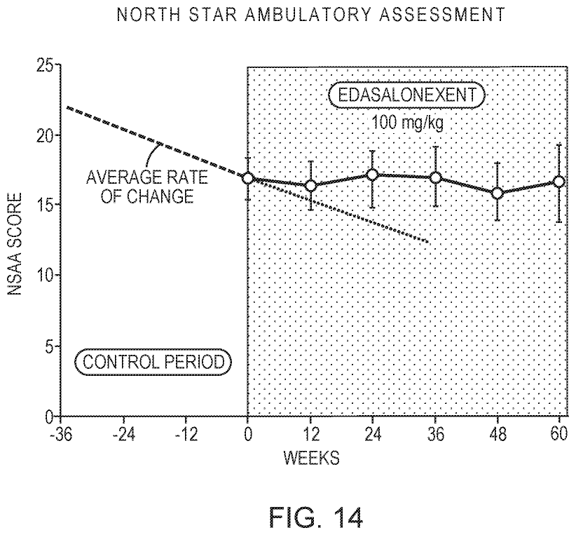

The North Star Ambulatory Assessment (NSAA) is a validated functional scale specifically designed for ambulant boys with DMD and measures overall function in young boys. As shown in FIG. 14, disease progression was slowed in study subjects during the 100 mg/kg/day treatment period (60 weeks) as measured by NSAA scores which stabilized compared to the rate of change of NSAA scores during the off-treatment control period (36 weeks prior to edasalonexent dosing period).

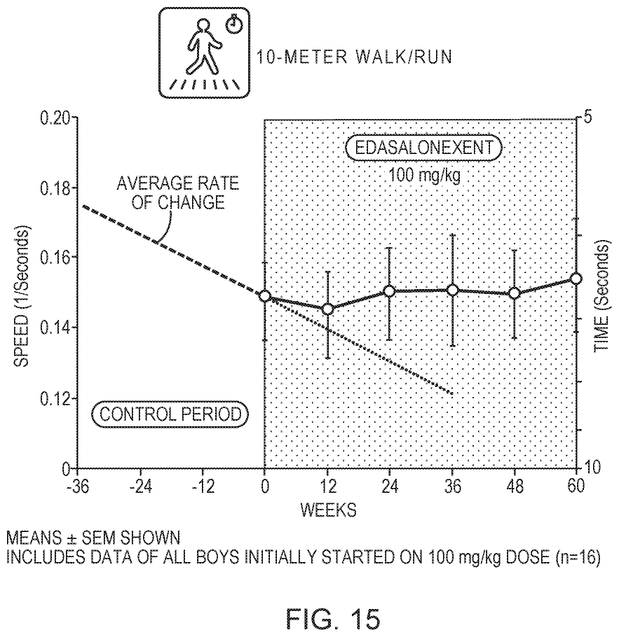

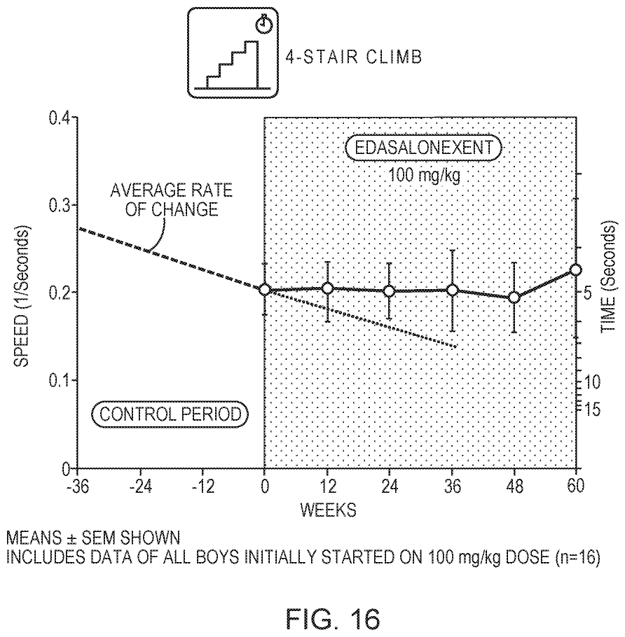

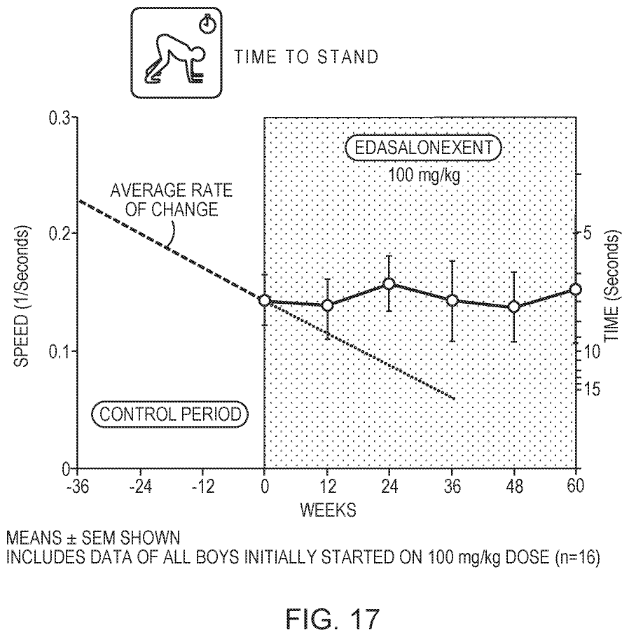

The 10-meter walk/run, 4-stair climb, and time to stand are all timed tests used as a measure of function in boys with DMD. In each of these tests, as shown in FIGS. 15-17, the average rate of change of speed by study subjects in completing these physical tasks stabilized during the 60 week 100 mg/kg/day edasalonexent treatment period as compared with the rate of change during the off-treatment control period (36 weeks preceding the treatment period). All timed function test values stabilized during the treatment period.

Accordingly, these results suggest that treatment of DMD patients with 100 mg/kg/day in three 33 mg/kg doses provides a clinically meaningful slowing of disease progression over the 60 week treatment period as compared to the off-treatment control period.

### 4.D Edasalonexent Significantly Improved the Rate of Change of MRI T2 Levels in DMD Study Subjects

The MR transverse relaxation time constant assessed by MRI (MRI-T₂; also referred to as bulk T₂) is sensitive to several pathophysiological features of disease pathology in DMD, including muscle damage, inflammation, and fat infiltration and provides a method for monitoring disease pathology over a wide age range. Furthermore, the proportion of fat infiltration in the muscle (fat fraction), measured using the gold standard, proton MRS (¹H-MRS), or a 3-point Dixon imaging technique, is associated with disease progression and correlates with performance on functional tests. (Willcocks et al., (2016), ANN. NEUROL., 79(4):535-47; Barnard et al., (2018), PLoS ONE, 13(3):E0194283). These are non-invasive measures of disease progression in DMD that are elevated and increase with age; changes in these measures strongly correlate with changes in function and predict future loss of functional milestones.

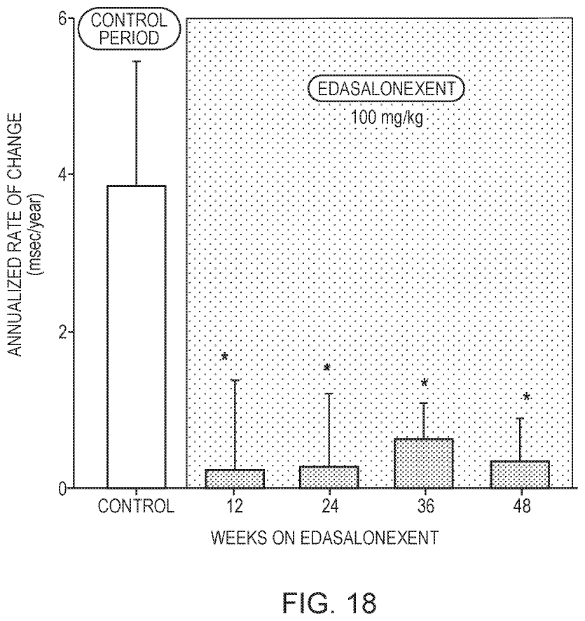

As shown in FIG. 18, the annualized rate of change (msec/year) of MRI-T2 values in study subjects during the edasalonexent treatment period of 100 mg/kg/day (60 weeks) was significantly improved over the rate of change during the off-treatment period (36 weeks prior to edasalonexent treatment period). The MRI-T2 value was a composite of 5 lower leg muscles. Stabilization of MRI-T2 values is consistent with the concomitant slowing of DMD disease progression observed through the functional assessments discussed previously.

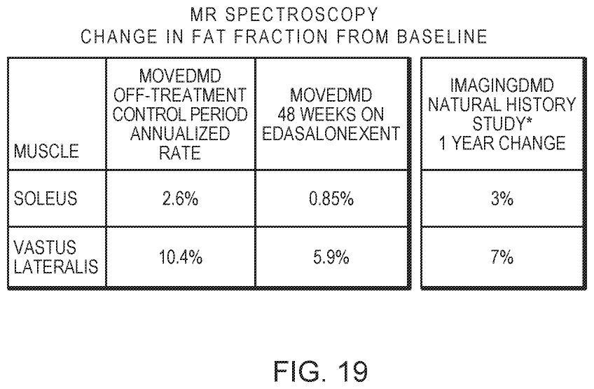

Further, as shown in FIG. 19, the change in fat fraction in study subjects as assessed through MR spectroscopy after 48 weeks receiving 100 mg/kg/day was 0.85% for the soleus (calf muscle), and 5.9% for the vastus lateralis (a quadriceps muscle), whereas during the 26 week off-treatment control period prior to edasalonexent administration, change in fat fraction were 2.6% for the soleus and 10.4% for the vastus lateralis. Accordingly, edasalonexent at 100 mg/kg/day was effective in decreasing the rate of increase in fat fraction in these muscles as compared to the off-treatment period. This is an improvement also over the observed changes in fat fraction in the ImagingDMD natural history study where the 1 year change was 3% for the soleus and 7% for the vastus lateralis (despite greater than 75% of the study subjects being on chronic steroids).

Accordingly, these MRI measures support positive edasalonexent treatment effects over 48 weeks at the 100 mg/kg/day (3 doses of 33 mg/kg/day) in boys afflicted with DMD.

4.E Edasalonexent was Well Tolerated with No Safety Signals

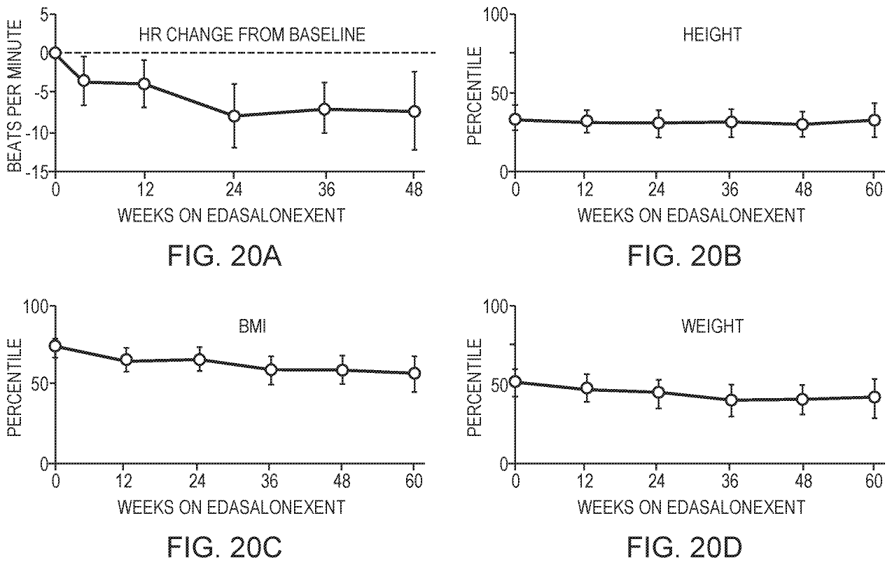

There were no adverse findings with respect to safety of edasalonexent administered at 100 mg/kg in the study subjects. The drug was well tolerated with the majority of adverse events being mild in nature and mostly gastrointestinal. No adverse trends in hematology, chemistry, renal or adrenal function, or calcium or phosphate levels were observed. As shown in FIG. 20, study subjects' heart rates decreased toward age normative values and age appropriate increases in height and weight were observed as shown by the steady percentiles for height, weight, and body mass index (BMI) over the treatment period based on percentiles per the CDC Growth Chart for boys of similar age.

### 4.F Conclusions

These data demonstrate that sustained exposure and time over threshold are drivers for pharmacodynamic signal and efficacy, that sustained exposure above a threshold level can be achieved with 100 mg/kg/day in three 33mg/kg doses, and that this dosing shows clinically meaningful slowing of disease progression over 1 year compared to the off-treatment control period in study subjects.

### Example 5: Dosing Regimen of Edasalonexent for Child with DMD

A child weighing 20 kg and suffering from DMD may be prescribed a daily dose of 100 mg/kg of edasalonexent, resulting in a total dose of 2,000 mg. This total dose is divided into three doses. The first dose is taken orally with breakfast, the second with lunch, and the third with dinner. All doses are taken with food containing at least 8 g of fat to aid absorption. While it is desirable to divide the dose into three equal doses, because edasalonexent is available in 250 mg capsules, this is not possible. In order to minimize the number of capsules it may be desirable to administer the 2,000 mg dose as 750 mg at breakfast (3×250 mg capsules), 500 mg at lunch (2×250 mg capsules), and 750 mg (3×250 mg capsules) at dinner. If the 3 doses are uneven, then the larger dose or doses may preferably be administered at breakfast or dinner and the smaller dose be given at lunch time.

A child weighing 28 kg and suffering from DMD may be prescribed a daily dose of 100 mg/kg of edasalonexent, resulting in a total dose of 2,800 mg. This total dose is divided into three doses. The first dose is taken orally with breakfast, the second with lunch, and the third with dinner. All doses are taken orally with food containing at least 8 g of fat to aid absorption. While it is desirable to divide the dose into three equal doses, because edasalonexent is available in 250 mg capsules, this is not possible. Accordingly, the 2,800 mg dose can be increased to 3000 mg and administered as 1,000 mg at breakfast (4×250 mg capsules), 1,000 mg at lunch (4×250 mg capsules), and 1,000 mg (4×250 mg capsules) at dinner. The total dose is therefore 3,000 mg because of the amount of edasalonexent provided in the capsules. However, the total actual dose (3,000 mg/day) does not exceed more than 10% of the recommended dose, i. e. , 2800 mg/day. In this case, the actual dose is 7.1% greater than the recommended dose. In an alternative approach, the fourth 250 mg capsule at lunch may be replaced with a 100 mg capsule so that the child received 1,000 mg with breakfast, 850 mg with lunch, and 1,000 mg with dinner to give a total daily dose of 2850 mg, in which case the actual dose is 1.8% greater than the 2800 mg recommended dose.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A method of treating muscular dystrophy in a subject in need thereof, the method comprising administering to the subject a dosing regimen of a compound having the structure of Formula I, 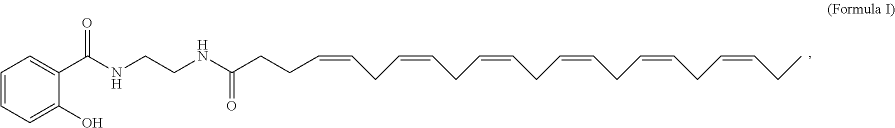 [SMILES: CC/C=C\C/C=C\C/C=C\C/C=C\C/C=C\C/C=C\CCC(=O)NCCNC(=O)c1ccccc1O]
or a pharmaceutically acceptable salt thereof, effective to achieve a threshold plasma concentration of the compound in the subject of at least about 20 ng/ml for least 12 hours in a 24 hour period.

2. The method of claim 1, wherein the threshold plasma concentration is from about 20 ng/ml to about 200 ng/ml.

3. The method of claim 1 or 2, wherein the compound is at or above the threshold concentration for at least about 13 hours, about 14 hours, about 15 hours, about 16 hours, about 17 hours, about 18 hours, about 19 hours, about 20 hours, about 21 hours, about 22 hours, about 23 hours, or about 24 hours in a 24 hour period.

4. The method of any one of claims 1-3, wherein the dosing regimen comprises one, two or three doses of the compound per day.

5. The method of claim 4, wherein each dose comprises from about 25 mg/kg to about 100 mg/kg of the compound.

6. The method of any one of claims 1-5, wherein the total daily dosage comprises from about 100 mg/kg to about 200 mg/kg, or from about 100 mg/kg to about 150 mg/kg.

7. The method of claim 6, wherein the total daily dosage comprises about 133 mg/kg.

8. The method of claim 6, wherein the total daily dosage comprises about 100 mg/kg.

9. The method of claim 6, wherein the total daily dosage comprises from about 90 mg/kg to about 110 mg/kg.

10. The method of claim 6, wherein the total daily dosage comprises 100 mg/kg±5%, 100 mg/kg±10%, 100 mg/kg±15%, or 100 mg/kg±20%.

11. The method of any one of claims 1-10, wherein the dosing regimen comprises three doses per day.

12. The method of claim 11, wherein the three doses comprise equal amounts of the compound.

13. The method of claim 11 or 12, wherein each dose comprises from about 25 mg/kg to about 50 mg/kg of the compound.

14. The method of claim 11 or 12, wherein each doses comprises from about 20 mg/kg to about 40 mg/kg.

15. The method of any one of claims 12-14, wherein each dose comprises about 33 mg/kg of the compound.

16. The method of claim 11, 13 or 14, wherein the first dose and the second dose comprises a smaller amount of the compound than the third dose.

17. The method of any one of claims 11-15, wherein the three doses are equal and are administered in dosage forms that contain 250 mg or 100 mg of the compound of Formula I.

18. The method of claim 17, wherein the three doses equal a total daily dose of 100 mg/kg±5%, 100 mg/kg±10%, 100 mg/kg±15%, or 100 mg/kg±20%.

19. The method of claim 17 or 18, wherein the total daily dose does not exceed 6,000 mg.

20. The method of any one of claims 11-19, wherein the first dose is administered in the morning, the second dose is administered at mid-day, and the third dose is administered in the evening.

21. The method of any one of claims 4-20, wherein each dose is administered with food.

22. The method of claim 20, wherein each dose is administered at the time of a meal.

23. The method of claim 20, wherein the first dose is administered at the time of breakfast, the second dose is administered at the time of lunch, and the third dose is administered at the time of dinner.

24. The method of claim 23, wherein two doses are administered with breakfast and dinner that are larger than the dose administered with lunch.

25. The method of any one of claims 4-24, wherein the dose is taken with food containing at least 8 g of fat.

26. The method of any one of claims 1-25, wherein the compound is administered in a pharmaceutical composition.

27. The method of claim 26, wherein the composition further comprises one or more of glyceryl monooleate (type 40), polysorbate 80, polyethylene glycol 400, or DL-α-tocopherol.

28. The method of claim 26, wherein the composition comprises 50-70% by weight of the compound.

29. The method of any one of claims 26-28, wherein the composition is formulated as a capsule.

30. The method of any one of claims 1-29, wherein the compound is administered orally.

31. The method of any one of claims 1-30, wherein the method reduces inflammation in quadriceps muscle by at least 20%.

32. The method of any one of claims 1-31, wherein the method reduces fibrosis in quadriceps muscle by at least 20%.

33. The method of any one of claims 1-32, wherein the muscular dystrophy is Duchenne muscular dystrophy (DMD).

34. The method of any one of claims 1-33, wherein the subject is human.

35. A pharmaceutical composition comprising 50-70% by weight of a compound having the structure of Formula I, 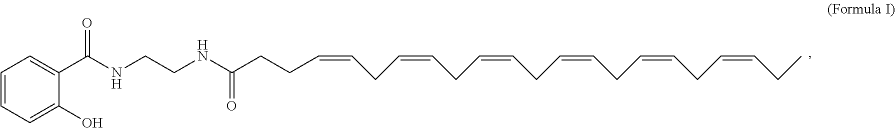 [SMILES: CC/C=C\C/C=C\C/C=C\C/C=C\C/C=C\C/C=C\CCC(=O)NCCNC(=O)c1ccccc1O]
or a pharmaceutically acceptable salt thereof, and optionally one, two, three, or four of: a solvent or diluent; a surfactant; a co-solvent; and an anti-oxidant .

36. The pharmaceutical composition of claim 35, wherein the solvent or diluent is glyceryl monooleate (type 40).

37. The pharmaceutical composition of claim 35 or 36, wherein the surfactant is a non-ionic surfactant.

38. The pharmaceutical composition of claim 37, wherein the non-ionic surfactant is polysorbate 80.

39. The pharmaceutical composition of any one of claims 35-38, wherein the co-solvent is polyethylene glycol 400.

40. The pharmaceutical composition of any one of claims 35-39, wherein the anti-oxidant is DL-α-tocopherol.
